SEQUENCE LISTING (12 sequences)

SEQ ID NO: 1 - protein, 12 aa, Artificial Sequence
  C-terminal sequence 1
ESKYGPPCPPCP

SEQ ID NO: 2 - protein, 13 aa, Artificial Sequence
  C-terminal sequence 2
ESKYGPPCPPCPA

SEQ ID NO: 3 - protein, 17 aa, Artificial Sequence
  C-terminal sequence 3
ESKYGPPCPPCPAPEAA

SEQ ID NO: 4 - protein, 18 aa, Artificial Sequence
  C-terminal sequence 4
GGGGSAESKYGPPCPPCP

SEQ ID NO: 5 - protein, 19 aa, Artificial Sequence
  C-terminal sequence 5
GGGGSAESKYGPPCPPCPA

SEQ ID NO: 6 - protein, 23 aa, Artificial Sequence
  C-terminal sequence 6
GGGGSAESKYGPPCPPCPAPEAA

SEQ ID NO: 7 - protein, 8 aa, Artificial Sequence
  C-terminal sequence 7
GPPCPPCP

SEQ ID NO: 8 - protein, 9 aa, Artificial Sequence
  C-terminal sequence 8
GPPCPPCPA

SEQ ID NO: 9 - protein, 13 aa, Artificial Sequence
  C-terminal sequence 9
GPPCPPCPAPEAA

SEQ ID NO: 10 - protein, 454 aa, Artificial Sequence
  anti TNF heavy chain 1
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 11 - protein, 473 aa, Artificial Sequence
  anti TNF heavy chain 2
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGGGGSGGGGSGGGGSAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 12 - protein, 19 aa, Artificial Sequence
  GS linker
GGGGGGGSGGGGSGGGGSA

(19) United States
(12) Patent Application Publication
(10) Pub. No.: US 2024/0043522 A1
(43) Pub. Date: Feb. 8, 2024

(54) EFFECTORLESS FC MOLECULES

(71) Applicant: SANOFI, Paris (FR)

(72) Inventors: Werner DITTRICH, Frankfurt am Main (DE); Marcus Hermann KORN, Frankfurt am Main (DE); Christian LANGE, Frankfurt am Main (DE); Thomas LANGER, Frankfurt am Main (DE); Ziyu LI, Frankfurt am Main (DE); Ercole RAO, Frankfurt am Main (DE)

(21) Appl. No.: 18/268,840

(22) PCT Filed: Dec. 20, 2021
(86) PCT No.: PCT/EP2021/086735

§ 371 Date: Jun. 21, 2023

(30) Foreign Application Priority Data
Dec. 22, 2020 (EP) 20315503.1

(51) Int. Cl. C07K 16/24

(52) U.S. Cl. CPC C07K 16/241; C07K 2317/71; C07K 2317/524; C07K 2317/53

(57) ABSTRACT

The present invention provides molecules comprising a modified Fc region which do not mediate antibody effector functions such as antibody-dependent cellular cytotoxicity (ADCC) but maintain a long serum half-life due to binding to the neonetal Fc receptor (FcRn). To this end, the molecules of the present invention do not comprise the disulfide bridges of the antibody hinge region but are linked C-terminally by at least two covalent bonds. Furthermore, the molecules of the present invention comprise CH2 domains with an additional disulfide bond.

### BACKGROUND

Monoclonal antibodies as well as Fc fusion proteins are very promising biopharmaceutical agents and the market for these compounds has increased significantly within the recent years. Reasons for the success of these molecules are their high specificity, safety and long half-lives in the circulation compared to small chemical compounds. Until 2017 more than 10 Fc fusion proteins have been approved and more are under review. Antibodies are composed of two different protein chains, a light chain (LC) and a heavy chain (HC). One LC is covalently linked to one HC and the HCs are on their parts covalently linked by disulfide bonds. The LC is composed of a variable domain (VL) and a constant domain (CL), whereas the HC consist of a variable domain (VH) and several constant domains (e.g., CH1, CH2 and CH3 domains for IgG). Functionally, an antibody can be divided into two Fab (Fragment antigen binding) regions and one Fc (Fragment crystallizable) region. Each Fab region consists of four domains: VL, CL, VH and CH1. The Fc region comprises the remaining constant domains (CH2 and CH3 domains for IgG). The Fab regions are connected to the Fc region via a flexible sequence, called the hinge region. This hinge region comprises disulfide bridges which link the HCs.

Antibodies are multifunctional proteins. While antigens are bound by Fab regions, effector functions of antibodies are mediated by the Fc region. The effector functions of antibodies include antibody-dependent cellular cytotoxicity (ADCC), antibody-dependent cellular phagocytosis (ADCP) and antibody-dependent complement deposition (ADCD). These effects are mediated via binding of the antibody Fc region to Fc gamma receptors (FcγRs) and the complement protein C1q, respectively. These antibody effector functions are important for the efficacy of natural antibodies and also some engineered therapeutic antibodies, e.g., in oncology. On the other hand, the effector functions are undesired for many other engineered therapeutic antibodies, e.g. antibodies against soluble targets such as pro-inflammatory cytokines. Such antibodies are often designed to act mainly or only by blocking their specific targets via binding of the Fab fragment, while any effector function of the Fc region elicits an interfering and potentially harmful immune reaction, at the extreme a “cytokine storm” as observed for the anti-CD28 antibody TGN1412.

On the other hand, the Fc region markedly prolongs half-life of antibodies and Fc fusion proteins in the circulation via a “recycling” mechanism mediated by the neonatal Fc-receptor (FcRn). Moreover, the Fc region contributes to the overall stability of the molecule and another important and desired function of the Fc-domain is its strong and specific binding to protein A.

Binding of antibodies and Fc fusion proteins to immobilized protein A is usually the first purification step during the purification of antibodies and Fc fusion proteins. Accordingly, a simple removal of the Fc region from the antibody molecule would have severe drawbacks.

Therefore, several variants Fc regions have been developed to reduce antibody effector functions while maintaining the positive effects of the Fc region (half-life prolongation, protein A binding, stabilization). These approaches include the aglycosylated variants and variants with point mutations (Wang et al., Protein Cell. 2018; 9 (1):63-73). However, many of the point mutation variants retains at least some antibody effector functions, while aglycosylated variants exhibit reduced solubility or stability (Dumet et al., MAbs. 2019; 11 (8): 1341-1350). Another attempt to abolish antibody effector functions is the modification or deletion of the hinge region which contributes to the binding of FcγRs and C1q to the Fc region (Vidarsson et al., Front Immunol. 2014; 5:520). It was shown that such a “hinge-less” antibody displays virtually no FcγRs-mediated effector function (Valeich et al., Antibodies (Basel). 2020 December; 9 (4): 50). However, these constructs had reduced stability and compromised binding to the FcRn which will translate in shorter in vivo half-life.

Thus, there remains the need for antibodies or Fc fusion proteins which have no antibody effector functions but maintain high stability, uncompromised FcRn binding and protein A binding. The present invention addresses this need by providing an Fc region which lacks a functional hinge region but comprises at least two covalent bonds, which are located C-terminally to the Fc part, and an additional internal disulfide bond in the CH2 domain.

### SUMMARY OF THE INVENTION

In a first aspect, the present invention provides a molecule comprising two polypeptides,
wherein said polypeptides form an antibody Fc region,
wherein said two polypeptides are linked by at least two covalent bonds which are located C-terminally to the portion of each polypeptide which forms the Fc region,
wherein said two polypeptides are not linked by a disulfide bond located N-terminally to the portion of each polypeptide which forms the Fc region,
wherein each polypeptide comprises a CH2 domain which is part of the Fc region, wherein each of these CH2 domains comprises an additional internal disulfide bond which is not present in the corresponding wild-type CH2 domain,
wherein the additional internal disulfide bond is selected from the group consisting of disulfide bonds formed between cysteine residues at the following positions according to EU numbering: P238C and D265C; P238C and L328C; S267C and A327C; and V240C and I332C.

In some embodiments, the additional internal disulfide bond is selected from the group consisting of disulfide bonds formed between cysteine residues at the following positions according to EU numbering: P238C and L328C; S267C and A327C; and V240C and I332C.

In some embodiments, the additional internal disulfide bond is selected from the group consisting of disulfide bonds formed between cysteine residues at the following positions according to EU numbering: P238C and L328C; and S267C and A327C.

In some embodiments, the two covalent bonds are two disulfide bonds.

In some embodiments, the at least two covalent bonds located C-terminally to the portion of each polypeptide which forms the Fc region are comprised in a sequence corresponding to the amino acid sequence of an antibody hinge region.

In some embodiments, the affinity to C1q and/or FcγR1, FcγR2 and/or FcγR3 is reduced at least 10-fold, compared to the same molecule comprising an antibody hinge region located N-terminally to the portion of each polypeptide which forms the Fc region.

In some embodiments, the affinity to protein A and/or FcgRn is not reduced, compared to the same molecule comprising an antibody hinge region located N-terminally to the portion of each polypeptide which forms the Fc region.

In some embodiments, the Fc region is the Fc region of IgG, IgM, IgA, IgD or IgE.

In some embodiments, the at least two covalent bonds are located C-terminally to a CH3 or CH4 domain on each polypeptide.

In some embodiments, the Fc region is the Fc region of an IgG antibody.

In some embodiments, the at least two covalent bonds are located C-terminally to a CH3 domain on each polypeptide.

In some embodiments, the molecule further comprises at least one active moiety.

In some embodiments, the active moiety is located N-terminal of the CH2 domain of at least one polypeptide and the molecule does not comprise an active moiety which is located C-terminal of the at least two covalent bonds which are located C-terminally to the portion of each polypeptide which forms the Fc region.

In some embodiments, the active moiety is an antigen-binding moiety.

In a second aspect, the present invention provides a polynucleotide encoding a molecule according to any embodiment of the first aspect.

The present invention provides molecules which do not display the potentially detrimental antibody effector functions (ADCC, ADCP, ADCD) of an Fc, but maintain all desired Fc functionalities: a high serum half-life via FcRn binding, easy purification via protein A binding and high molecule stability.

These advantageous properties of the molecules of the present invention are due to the combination of the following features: a lack of a functional antibody hinge region, which diminishes antibody effector functions, and the at least two C-terminal covalent bonds, as well as an additional internal disulfide bond in the CH2 domain, which both enhances stability. Thus, the present invention provides molecules with no antibody effector functions, uncompromised FcRn binding and enhanced stability.

Compared to known Fc variants with point mutations which diminish antibody effector functions (e.g. the “LALA” mutation (L234A and L235A), the lack of an N-terminal hinge region, as in the molecules of the present invention, abolishes ADCD more effectively (FIG. 8).

While hinge-less antibodies without C-terminal covalent bonds have a reduced stability and compromised FcRn binding (as shown in Valeich et al., Antibodies (Basel). 2020 December; 9 (4): 50), the molecules of the present invention are not compromised in FcRn binding.

In addition, a combination of the C-terminal covalent bonds and the additional internal disulfide bond in the CH2 domains of molecules of the present invention stabilize the molecule and can thus fully compensate for the lack of an N-terminal hinge region (Table 6).

The CH2 domains of the molecule comprise an additional internal disulfide bond which is not present in the corresponding wild-type CH2 domain. This further enhances the stability of the molecule. Certain internal disulfide bonds provide a better stabilization than others. In terms of stabilization, particularly favorable internal disulfide bonds are P238C/L328C, S267C/A327C and V240C/I332C.

Moreover, the two polypeptides of the molecules of the present invention form stable dimers due to the C-terminal at least two inter-chain covalent bonds. The molecules adopt proper folding, no aggregation mediated by the additional C-terminal covalent bonds was observed (see SEC-MALS data, FIGS. 4 and 11). Preventing aggregate formation is very important for molecules which comprise an Fc region because aggregates can mediate ADCD. Moreover, the C-terminal covalent bonds of molecules of the present invention mask the natural Fc C-terminus of the antibody which also prevents ADCD mediated by Fc Terminus aggregation.

Furthermore, the molecules have a high expression level in standard protein expression systems.

In some embodiments, the at least two C-terminal covalent bonds are comprised in an amino acid sequence which corresponds to the amino acid sequence of an antibody hinge region. In this case, the at least two C-terminal covalent bonds are at least two disulfide bonds within this sequence. Surprisingly, the sequence of the antibody hinge region forms inter-chain disulfide bonds also if positioned C-terminally to the Fc part. As the sequence of the antibody hinge region can be derived from a human endogenous antibody hinge region, the usage of exogeneous sequences can be avoided which reduces the risk of anti-drug antibody (ADA) formation.

In some embodiments, the molecule comprises an active moiety located N-terminally of the Fc part. In some embodiments, the molecule does not comprise an active moiety located C-terminally of the at least two covalent bonds which are located C-terminally to the Fc region. This has the advantage that the C-terminal part of the Fc cannot interfere with the active moiety, for instance does not hinder its binding to a target. In some embodiments, the active moiety is an antigen binding moiety.

### BRIEF DESCRIPTION OF THE FIGURES

FIG. 1. Schematic view of an IgG with indicated binding sites for Fc-binding proteins (A). FcγR and C1q are binding to the lower hinge region (Vidarsson et al., Front Immunol. 2014; 5:520). This site is distinct from the binding site of the FcRn and Protein A which is located at the lower CH2 and upper CH3 domain (Vidarsson et al., Front Immunol. 2014; 5:520). (B) Schematic view of an effector-less Fc region according to the present invention. The hinge region, located N-terminally of the Fc region, was removed, its sequence was shifted to the C-terminus of the Fc region. Moreover, the CH1 domain was connected to the CH2 domains via a linker sequence. Consequently, this new arrangement completely eliminates the binding sites for FcγRs and C1q.

FIG. 2. Overview of the different anti-TNFα antibody variants. The Fab is derived from adalimumab and is the same in all variants. Mab01 is adalimumab with IgG1 wild-type (wt). Mab02 is an IgG1 variant that has the known “LALA” (L234A/L235A) mutations as well as the N297A mutation to avoid glycosylation (Wang et al., Protein Cell. 2018; 9 (1):63-73). Mab03 has the IgG2 Fc-backbone which has a reduced effector functions compared to IgG1. Mab04 has an IgG1 Fc-backbone with the known E269R and K322A mutations. Mab05 has an IgG1 Fc-backbone with the LALA (L234A/L235A) mutation and additional the known P329A mutation. Mab06 is an Fc IgG1 according to the present invention (labeled as “upside-down”) with the sequence of the hinge region transferred to the C-terminus and no linker sequence between CH1 and CH2 domains. Mab07 is an Fc IgG1 according to the present invention (labeled as “upside-down”) further including a 19GS-linker between CH1 and CH2 domains.

FIG. 3. SDS-PAGE of purified antibody variants according to FIG. 2 under non-reducing and reducing conditions. M: Protein marker.

FIG. 4. Analytical size exclusion chromatography and calculated molecular masses of the different antibodies. (A) The relative signal intensities of the Rayleigh ratio, as a measure of the scattered light intensity, are plotted versus the elapsed time. (B) Close up view of the peaks. The calculated molecular masses of the proteins within the main peak-fraction are indicated. Mab06 is labeled “ud-IgG1”; Mab07 is labeled “ud-IgG1 (19GS).

FIG. 5. Binding of TNFα to the different antibodies as determined by SPR. Antibodies were immobilized on the chip and human TNFα was used as analyte. The corresponding antibody variants are indicated. Mab06 is labeled “ud-IgG1”; Mab07 is labeled “ud-IgG1 (19GS).

FIG. 6. SPR-affinity measurements of the different antibodies to different FcRs. For interaction analysis the FcRs were immobilized on the sensor chip. The sensograms show the binding profiles of the different antibodies at different concentrations. Mab06 is labeled “ud-IgG1”; Mab07 is labeled “ud-IgG1 (19GS).

FIG. 7. Effector function: ADCC. (A) Schematic drawing of the assay principle of the assay. Upon binding of a membrane-standing antigen an effector cell is activated upon FcγRIII activation. Activation of the effector cell leads to cell lysis of the target cell. ADCC assay results obtained with (B) IgG1 wt, IgG1 LALA N297A (aglycosylated), and IgG1 LALA P329A variant. (C) IgG1 wt, IgG1 E269R, K322A, and IgG2. (D) IgG1 wt, Fc IgG1 according to the present invention (“ud-IgG1”), Fc IgG1 according to the present invention including a 19GS-linker (“ud-IgG1 (19GS)”). Activities are given in % specific lysis (see “Methods” in the Examples section).

FIG. 8. Effector function: ADCD. (A) Schematic drawing of the assay principle of the assay. Formation of antigen-antibody complexes (immune complex) leads to recruitment of the complement system. The result is the assembly of the membrane attack complex which kills the target cell. (B) IgG1 wt, IgG1 LALA N297A (aglycosylated), and IgG1 LALA P329A variant. (C) IgG1 wt, IgG1 E269R, K322A, and IgG2. (D) IgG1 wt, Fc IgG1 according to the present invention (“ud-IgG1”), Fc IgG1 according to the present invention including a 19GS-linker (“ud-IgG1 (19GS)”). Activities are given in % specific lysis (see Materials & Methods).

FIG. 9. Overview of the different GLP1-RA-Fc fusion proteins. All fusion proteins contain the same GLP1-sequence and linker sequence to an Fc-domain. Note that GLP1-Fc01 and GLP1-Fc04 contain an IgG4 derived Fc-domain. This Fc variant contains the known F234A and L235A mutations for reduced effector function and the known S228P mutation to avoid half-antibody formation. All IgG1-Fc variants contain the LALA (L234A/L235A) mutations for reduced effector function. For sake of clarity, these mutations are not depicted in the figure. Fc variants according to the present invention (GLP-Fc04-GLP-Fc014 are labeled as “upside-down”.

FIG. 10. SDS-PAGE of purified GLP1-RA-Fc fusion proteins variants according to FIG. 9 under non-reducing (A) and reducing conditions (B). M: Protein marker.

FIG. 11. Analytical size exclusion chromatography and calculated molecular masses of the different GLP1-RA Fc fusion proteins. For clarity, only seven data sets are shown in one graph. (A, B) The relative signal intensities of the Rayleigh ratio are plotted versus the elapsed time. (C, D) Close up view of the peaks. The calculated molecular masses of the proteins within the main peak-fraction are indicated.

FIG. 12. Stability of GLP1-RA Fc fusion proteins as determined with nanoDSF. Maximum four variants are shown in one graph for clarity. (A) Variants GLP1-Fc01-GLP1-Fc04, (B) GLP1-Fc05-GLP1-Fc08, (C) GLP1-Fc09-GLP1-Fc12, and (D) GLP1-Fc13 and GLP1-14. All measurements were done in duplicates. Curve represents mean data and the standard deviation is indicated as shadow. The fluorescence intensity in each sample is measured at 330 nm and 350 nm. In each upper panel the ratio of the fluorescence intensity at 350 nm/330 nm is plotted versus the temperature. The 350 nm/330 nm ratio is a measure for a spectral shift of tryptophan-residues induced upon unfolding of the protein. In the lower panels the first derivatives of the melting curves are shown. The maxima of the first derivative were used to calculate the inflection points (IP) as a measure of protein stability.

FIG. 13. Pharmacodynamic profile of GLP1-Fc01 and GLP1-Fc04. Female db/db mice or lean controls were treated every fourth day s.c. for 14 days with 10 nmol/kg GLP1-Fc01 or GLP1-Fc03 vs. vehicle (PBS) or control. Body weight development (A) and fed blood glucose levels (B) was analyzed throughout the study, shown as glucose change from baseline (day 1). Timepoints of s.c. compound administration is indicated by arrows. Data are means+SEM, n=8 per group.

### DETAILED DESCRIPTION

The present invention provides molecules comprising two polypeptides, wherein said polypeptides form an antibody Fc region, wherein said two polypeptides are linked by at least two covalent bonds which are located C-terminally to the portion of each polypeptide which forms the Fc region, wherein said two polypeptides are not linked by a disulfide bond located N-terminally to the portion of each polypeptide which forms the Fc region, wherein each polypeptide comprises a CH2 domain which is part of the Fc region, wherein each of these CH2 domains comprises an additional internal disulfide bond which is not present in the corresponding wild-type CH2 domain, wherein the additional internal disulfide bond is selected from the group consisting of disulfide bonds formed between cysteine residues at the following positions according to EU numbering: P238C and D265C; P238C and L328C; S267C and A327C; and V240C and I332C.

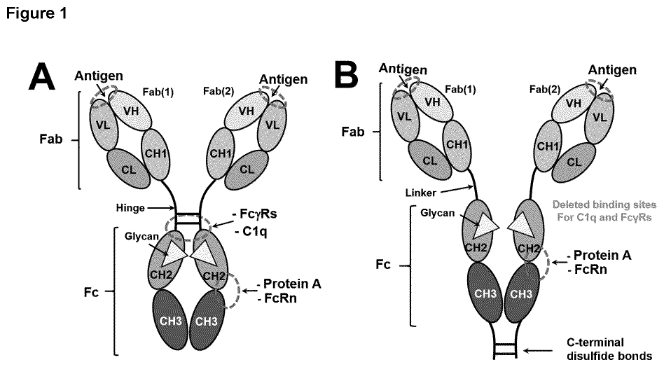

The molecule design of the present invention enables molecules which do not elicit the Fc-mediated antibody effector functions (ADCC, ADCP, ADCD), but maintain the desired function of the Fc region: FcRn binding, protein A binding and stabilization. The invention is based on the discovery that the binding site for FcγRs and C1q, which are responsible for the antibody effector functions, are partially located in the antibody hinge region while the binding site for FcRn and protein A are located in the more C-terminal part of the Fc region (FIG. 1). Thus, removal of the hinge region abolishes antibody effector functions without impairing the desired Fc functions. Moreover, two C-terminal covalent bonds and an additional internal disulfide bond in the CH2 domain of the molecules of the invention overcome the limitations (compromised FcRn binding, reduced stability) observed for a plain hinge-less antibody variant (Valeich et al., Antibodies (Basel). 2020 December; 9 (4): 50).

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions, etc.), whether supra or infra, are hereby incorporated by reference in their entirety.

As used in this description and the appended claims, the singular forms “a”, “an”, and “the” include plural referents, unless the content clearly dictates otherwise.

Antibody positions: The position of amino acids within antibody molecules or fragments of antibody molecules are specified according to EU numbering, unless indicated otherwise.

An “molecule”, as used herein, is group of atoms which are held together either by covalent interactions and/or by non-covalent interactions (e.g. hydrophobic or electrostatic interactions). In one embodiment, all atoms of the molecule are connected by covalent interactions.

In some embodiments, the molecule is an antibody. In some embodiments, the molecule is a multispecific antibody. In some embodiments, the molecule is an Fc fusion protein. In some embodiments, the molecule of the is a Fc fusion protein which comprises an Fc region and an active moiety.

A “polypeptide”, as used herein, is a chain of 10 or more amino acids which are linked covalently by peptide bonds. Accordingly, the term “polypeptide” can stand for a chain of a multi-chain protein, independent of the length of such chain. In some embodiments, a polypeptide is a chain of a multi-chain protein.

An “Fc region”, as used herein, is a fragment of an immunoglobulin molecule which is formed by several constant heavy chain (CH) immunoglobulin domains of two polypeptides. In natural antibodies, the Fc region mediates binding to Fc receptors and components of the complement system. For natural IgG, IgA and IgD, the Fc region is formed by the CH2 and CH3 domains of both heavy chains. For natural IgM and IgE, the Fc region is formed by the CH2, CH3 and CH4 domains of both heavy chains. The Fc regions of the present invention can be formed by the same CH domains as the Fc region of natural antibodies, i.e. CH2 and CH3 domains or CH2, CH3 and CH4 domains. The Fc regions of the present invention comprise CH domains which are either identical to CH domains of the natural immunoglobulins or are derived from CH domains of the natural immunoglobulins, e.g. by inserting one or several points mutations.

“hinge region”, as used herein, is a part of an antibody sequence which is located between the Fc region and the Fab regions. It provides segmental flexibility and can stabilize the antibody molecules. The hinge region can be clearly defined based on structural data, e.g. the IgG1 hinge region comprises the residues 221-237 (R. Nezlin, “The Immunoglobulins”, Academic Press, 1998, pages 23-26). In IgG antibodies, the hinge region comprises disulfide bonds which link the two heavy chains of the antibody. IgG1 and IgG4 comprise 2 such disulfide bonds, while IgG2 comprises 4 and IgG3 comprises 11 (Liu and May, 2012 MAbs. 2012 January-February; 4 (1): 17-23).

In the molecules of the present invention, a region is only designated as “hinge region” if it is located N-terminally of the Fc region, as it is the case for all natural antibodies. If an amino acid sequence which is identical or highly similar to a hinge region is placed C-terminally of the Fc region (as in some embodiments of molecules of the present invention), it is designated as “sequence corresponding to the amino acid sequence of an (antibody) hinge region”. Highly similar means at least 70% identity to the sequence of an antibody hinge region and/or comprising at least 7 contiguous amino acids of the sequence of an antibody hinge region.

A “covalent bond”, as used herein, is a chemical bond that involves the sharing of electron pairs between atoms. A covalent bond is distinct from non-covalent interaction, such as electrostatic interactions or hydrophobic effect.

The indication that a covalent bond, e.g. disulfide bonds, is “located C-terminally” of an Fc region means that, on both polypeptides forming said Fc region, the amino acid residues, e.g. cysteine residues, which are involved in the formation of said covalent bond, are located C-terminally of the portion of the polypeptides which form the Fc region.

The indication that no covalent bond, e.g. disulfide bond, is “located N-terminally” of an Fc region means that, on both polypeptides forming said Fc region, there are no amino acid residues, e.g. cysteine residues, which are involved in the formation of such a covalent bond, located N-terminally of the portion of the polypeptide which form the Fc region.

An “internal disulfide bond”, as used herein, is a disulfide bond formed by two cysteine residues within one immunoglobulin domain, e.g. a CH domain. This means that an internal disulfide bond is always an intra-chain disulfide bond, not an inter-chain disulfide bond which links different polypeptide chains. An “internal disulfide bond which is not present in the corresponding wild-type CH2 domain”, also called “additional internal disulfide bond” is an internal disulfide bond which is not present in the natural CH2 domain, i.e. the natural IgG, IgM, IgA, IgD or IgE CH2 domain, from which the modified CH2 of the invention was derived.

In some embodiments, the internal disulfide is selected from disulfide bonds formed between cysteine residues at the following positions: P238C and D265C; P238C and L328C; S267C and A327C; and V240C and I332C.

A “linker”, as used herein, is a short and flexible amino acid sequence which links two regions of a molecule. For instance, a linker can link a Fab region and the Fc region; or an active moiety and the Fc region; or the Fc region and the C-terminal region of the molecule which comprises the at least two inter-chain covalent bonds. A typical linker consists of 1-20 amino acids.

In some embodiments, the linker comprises several glycine and/or serine residues. In some embodiments, the linker consists of glycine and/or serine residues. In some embodiments, the linker comprises an amino acid sequence which correspond to an amino acid sequence of a natural antibody molecule. In some embodiments, the linker comprises an amino acid sequence which correspond to an amino acid sequence of the natural antibody molecule from which the CH2 and CH3 domains of the Fc region are derived.

In one embodiment, the Fc region and the C-terminal region which comprises the at least two inter-chain covalent bonds are linked by a linker which consists of 6 amino acids. In one embodiment, the Fc region and the C-terminal region which comprises the at least two inter-chain covalent bonds are linked by a linker which consists of 1 amino acid.

An “active moiety”, as used herein, is a molecule, or part of a molecule, which exerts a biological function by interacting with a biological structure (e.g., a receptor for said molecule).

In some embodiments, the active moiety is a peptide. In some embodiments, the active moiety is a receptor agonist or receptor antagonist. In some embodiments, the active moiety is an enzyme. In some embodiments, the active moiety is a cytokine. In some embodiments, the active moiety is an antigen-binding moiety.

An “antigen-binding moiety”, as used herein, is an antibody or antibody fragment which is capable of binding to the target of such antibody. Examples of antigen-binding moieties are a Fab fragment, a F(ab)₂ fragment, an scFv (single-chain variable fragment), an sdAb (single-domain antibody), a VHH, an ISVD (immunoglobulin single variable domain), (e.g., a NANOBODY molecule), and a VNAR (variable new antigen receptor).

### Fc Region

In some embodiments of the present invention, the two polypeptides form an Fc region of an IgG, IgM, IgA, IgD or IgE antibody. In some embodiments of the present invention, the two polypeptides form an Fc region of an IgG antibody. In some embodiments of the present invention, the two polypeptides form an Fc region of an IgG1 antibody. In some embodiments of the present invention, the two polypeptides form an Fc region of an IgG2 antibody. In some embodiments of the present invention, the two polypeptides form an Fc region of an IgG3 antibody. In some embodiments of the present invention, the two polypeptides form an Fc region of an IgG4 antibody. In some embodiments of the present invention, the two polypeptides form a mixed Fc region which comprises CH domains of different antibody classes or subclasses, e.g., IgG1 CH3 domains and IgG4 CH2 domains.

### C-Terminal Covalent Bonds

In some embodiments, the at least two covalent bonds are at least two disulfide bonds. In some embodiments, the at least two covalent bonds are two disulfide bonds.

In some embodiments, the at least two covalent bonds are comprised in a sequence corresponding to the amino acid sequence of an antibody hinge region. In some embodiments, the sequence corresponding to the amino acid sequence of an antibody hinge region is identical to the sequence of an antibody hinge region. In some embodiments, the sequence corresponding to the amino acid sequence of an antibody hinge region is highly similar to the sequence of an antibody hinge region. In some embodiments, the sequence has at least 70% identity to the sequence of an antibody hinge region. In some embodiments, the sequence has at least 75% identity to the sequence of an antibody hinge region. In some embodiments, the sequence has at least 80% identity to the sequence of an antibody hinge region. In some embodiments, the sequence has at least 90% identity to the sequence of an antibody hinge region. In some embodiments, the sequence comprises at least 7 contiguous amino acids of the sequence of an antibody hinge region. In some embodiments, the sequence comprises at least 10 contiguous amino acids of the sequence of an antibody hinge region. In some embodiments, the sequence comprises at least 15 contiguous amino acids of the sequence of an antibody hinge region.

In some embodiments, the sequence corresponding to the amino acid sequence of an antibody hinge region is identical or highly similar to the sequence of an IgG4 hinge region. In some embodiments, the sequence corresponding to the amino acid sequence of an antibody hinge region is selected from, ESKYGPPCPPCP (SEQ ID NO:1), ESKYGPPCPPCPA (SEQ ID NO:2), ESKYGPPCPPCPAPEAA (SEQ ID NO:3), GGGGSAESKYGPPCPPCP (SEQ ID NO:4), GGGGSAESKYGPPCPPCPA (SEQ ID NO:5) GGGGSAESKYGPPCPPCPAPEAA (SEQ ID NO:6). In some embodiments, the at least two covalent bonds are comprised in a proline-cysteine rich sequence. In some embodiments, said proline-cysteine rich sequence is selected from GPPCPPCP (SEQ ID NO:7), GPPCPPCPA (SEQ ID NO:8), GPPCPPCPAPEAA (SEQ ID NO:9).

In some embodiments, the at least two covalent bonds are covalent bonds created by click chemistry between non-natural amino acids.

In some embodiments, at least two covalent bonds are located C-terminally to a CH2, CH3 and/or CH4 domain on each polypeptide

In some embodiments, at least two covalent bonds are located C-terminally to a CH2 and/or CH3 domain on each polypeptide.

In some embodiments, at least two covalent bonds are located C-terminally to a CH2 and CH3 domain on each polypeptide.

### Internal Disulfide Bond in CH2 Domain

Each polypeptide comprises a CH2 domain which is part of the Fc region, wherein each of these CH2 domains comprises an internal disulfide bond which is not present in the corresponding wild-type CH2 domain. Said internal disulfide bond is selected from the group consisting of disulfide bonds formed between cysteine residues at the following positions: P238C and D265C; P238C and L328C; S267C and A327C; and V240C and I332C. In some embodiments, said internal disulfide bond is selected from the group consisting of disulfide bonds formed between cysteine residues at the following positions: P238C and L328C; S267C and A327C; and V240C and I332C. In some embodiments, said internal disulfide bond is selected from the group consisting of disulfide bonds formed between cysteine residues at the following positions: P238C and L328C; and S267C and A327C. In some embodiments, said internal disulfide bond is a disulfide bond formed between cysteine residues P238C and D265C. In some embodiments, said internal disulfide bond is a disulfide bond formed between cysteine residues P238C and L328C. In some embodiments, said internal disulfide bond is a disulfide bond formed between cysteine residues S267C and A327C. In some embodiments, said internal disulfide bond is a disulfide bond formed between cysteine residues V240C and I332C. Disclosed is also an internal disulfide bond which is a disulfide bond formed between cysteine residues P238C and A327C.

In some embodiments, each of the polypeptide comprises a linker, wherein the linker links the C-terminus of an immunoglobulin domain forming the Fc region to one of the disulfide bonds located C-terminally of the immunoglobulin domains. In some embodiments, said linker comprises 6 amino acids.

### Exemplary Embodiments

In some embodiments, the two polypeptides form an Fc region of an IgG antibody and the at least two covalent bonds are at least two disulfide bonds. In some embodiments, the two polypeptides form an Fc region of an IgG antibody and the at least two covalent bonds are two disulfide bonds. In some embodiments, the two polypeptides form an Fc region of an IgG antibody and the at least two covalent bonds are comprised in a sequence corresponding to the amino acid sequence of an antibody hinge region. In some embodiments, the two polypeptides form an Fc region of an IgG antibody and the at least two covalent bonds are comprised in a proline-cysteine rich sequence. In some embodiments, the two polypeptides form an Fc region of an IgG antibody and the at least two covalent bonds are covalent bonds created by click chemistry between non-natural amino acids.

In some embodiments, the two polypeptides form an Fc region of an IgG1 antibody and the at least two covalent bonds are at least two disulfide bonds. In some embodiments, the two polypeptides form an Fc region of an IgG1 antibody and the at least two covalent bonds are two disulfide bonds. In some embodiments, the two polypeptides form an Fc region of an IgG1 antibody and the at least two covalent bonds are comprised in a sequence corresponding to the amino acid sequence of an antibody hinge region. In some embodiments, the two polypeptides form an Fc region of an IgG1 antibody and the at least two covalent bonds are comprised in a proline-cysteine rich sequence. In some embodiments, the two polypeptides form an Fc region of an IgG1 antibody and the at least two covalent bonds are covalent bonds created by click chemistry between non-natural amino acids.

In some embodiments, the two polypeptides form an Fc region of an IgG4 antibody and the at least two covalent bonds are at least two disulfide bonds. In some embodiments, the two polypeptides form an Fc region of an IgG4 antibody and the at least two covalent bonds are two disulfide bonds. In some embodiments, the two polypeptides form an Fc region of an IgG4 antibody and the at least two covalent bonds are comprised in a sequence corresponding to the amino acid sequence of an antibody hinge region. In some embodiments, the two polypeptides form an Fc region of an IgG4 antibody and the at least two covalent bonds are comprised in a proline-cysteine rich sequence. In some embodiments, the two polypeptides form an Fc region of an IgG4 antibody and the at least two covalent bonds are covalent bonds created by click chemistry between non-natural amino acids.

In some embodiments, the two polypeptides form an Fc region of an IgG1 antibody and each of the CH2 domains of the Fc region comprises an internal disulfide bond which is not present in the corresponding wild-type CH2 domain. In some embodiments, the two polypeptides form an Fc region of an IgG1 antibody and each of the CH2 domains of the Fc region comprises an internal disulfide bond which is not present in the corresponding wild-type CH2 domain, wherein said internal disulfide bond is selected from the group consisting of disulfide bonds formed between cysteine residues at the following positions: P238C and D265C; P238C and L328C; S267C and A327C; and V240C and I332C. In some embodiments, the two polypeptides form an Fc region of an IgG1 antibody and each of the CH2 domains of the Fc region comprises an internal disulfide bond which is not present in the corresponding wild-type CH2 domain, wherein said internal disulfide bond is selected from the group consisting of disulfide bonds formed between cysteine residues at the following positions: P238C and L328C; S267C and A327C; and V240C and I332C.

In some embodiments, the two polypeptides form an Fc region of an IgG1 antibody, the at least two covalent bonds are at least two disulfide bonds, and each of the CH2 domains of the Fc region comprises an internal disulfide bond which is not present in the corresponding wild-type CH2 domain. In some embodiments, the two polypeptides form an Fc region of an IgG1 antibody, the at least two covalent bonds are two disulfide bonds, and each of the CH2 domains of the Fc region comprises an internal disulfide bond which is not present in the corresponding wild-type CH2 domain. In some embodiments, the two polypeptides form an Fc region of an IgG1 antibody, the at least two covalent bonds are comprised in a sequence corresponding to the amino acid sequence of an antibody hinge region, and each of the CH2 domains of the Fc region comprises an internal disulfide bond which is not present in the corresponding wild-type CH2 domain. In some embodiments, the two polypeptides form an Fc region of an IgG1 antibody, the at least two covalent bonds are comprised in a proline-cysteine rich sequence, and each of the CH2 domains of the Fc region comprises an internal disulfide bond which is not present in the corresponding wild-type CH2 domain. In some embodiments, the two polypeptides form an Fc region of an IgG1 antibody, the at least two covalent bonds are covalent bonds created by click chemistry between non-natural amino acids, and each of the CH2 domains of the Fc region comprises an internal disulfide bond which is not present in the corresponding wild-type CH2 domain.

In some embodiments, the two polypeptides form an Fc region of an IgG1 antibody, the at least two covalent bonds are at least two disulfide bonds, and each of the CH2 domains of the Fc region comprises an internal disulfide bond which is not present in the corresponding wild-type CH2 domain, wherein said internal disulfide bond is selected from the group consisting of disulfide bonds formed between cysteine residues at the following positions: P238C and L328C; S267C and A327C; and V240C and I332C. In some embodiments, the two polypeptides form an Fc region of an IgG1 antibody, the at least two covalent bonds are two disulfide bonds, and each of the CH2 domains of the Fc region comprises an internal disulfide bond which is not present in the corresponding wild-type CH2 domain, wherein said internal disulfide bond is selected from the group consisting of disulfide bonds formed between cysteine residues at the following positions: P238C and L328C; S267C and A327C; and V240C and I332C. In some embodiments, the two polypeptides form an Fc region of an IgG1 antibody, the at least two covalent bonds are comprised in a sequence corresponding to the amino acid sequence of an antibody hinge region, and each of the CH2 domains of the Fc region comprises an internal disulfide bond which is not present in the corresponding wild-type CH2 domain, wherein said internal disulfide bond is selected from the group consisting of disulfide bonds formed between cysteine residues at the following positions: P238C and L328C; S267C and A327C; and V240C and I332C. In some embodiments, the two polypeptides form an Fc region of an IgG1 antibody, the at least two covalent bonds are comprised in a proline-cysteine rich sequence, and each of the CH2 domains of the Fc region comprises an internal disulfide bond which is not present in the corresponding wild-type CH2 domain, wherein said internal disulfide bond is selected from the group consisting of disulfide bonds formed between cysteine residues at the following positions: P238C and L328C; S267C and A327C; and V240C and I332C. In some embodiments, the two polypeptides form an Fc region of an IgG1 antibody, the at least two covalent bonds are covalent bonds created by click chemistry between non-natural amino acids, and each of the CH2 domains of the Fc region comprises an internal disulfide bond which is not present in the corresponding wild-type CH2 domain, wherein said internal disulfide bond is selected from the group consisting of disulfide bonds formed between cysteine residues at the following positions: P238C and L328C; S267C and A327C; and V240C and I332C.

In some embodiments, the molecule is an antibody and comprises only endogenous sequences. In some embodiments, the molecule is a multispecific antibody and comprises only endogenous sequences. In some embodiments, the molecule is a monospecific antibody and comprises only endogenous sequences.

In some embodiments, the molecule comprises an active moiety. In some embodiments, the active moiety is an antigen-binding moiety. In some embodiments, the active moiety is a peptide. In some embodiments, the active moiety is a receptor agonist or receptor antagonist. In some embodiments, the active moiety is an enzyme. In some embodiments, the active moiety is a cytokine. In some embodiments, the active moiety is a chemokine. In some embodiments, the active moiety is a toxin. In some embodiments, the active moiety is a radiocontrast agent. In some embodiments, the active moiety is a nutrient.

In some embodiments, the molecule comprises an active moiety located N-terminally of the Fc part. In some embodiments, the molecule does not comprise an active moiety located C-terminally of the at least two covalent bonds which are located C-terminally to the Fc region. This has the advantage that the C-terminal part of the Fc cannot interfere with the active moiety, for instance does not hinder its binding to a target. In some embodiments, the active moiety is an antigen binding moiety.

In some embodiments, each polypeptide comprises an antigen-binding moiety located N-terminally of the Fc part. In some embodiments, each polypeptide comprises the heavy chain of a Fab fragment located N-terminally of the Fc part. In some embodiments, each polypeptide comprises an ISVD located N-terminally of the Fc part.

### Polynucleotides, Vectors, Cell

In a second aspect, the present invention relates to one or more polynucleotides encoding a molecule of the present invention. This refers to all embodiments described above. The one or more polynucleotides according to the second aspect may also encode the one or more additional polypeptides comprised in a complex with the molecule of the present invention. In one embodiment, the one or more polynucleotides encode an antibody or antibody-like structure. In one embodiment, the one or more polynucleotides are isolated.

In a third aspect, the present invention relates to one or more expression vectors comprising the one or more polynucleotides according to the second aspect of the invention. The term “vector” as used herein refers to any molecule (e.g., nucleic acid, plasmid, or virus) that is used to transfer coding information to a host cell. The term “vector” includes a nucleic acid molecule that is capable of transporting another nucleic acid to which it has been fused. One type of vector is a “plasmid,” which refers to a circular double-stranded DNA molecule into which additional DNA segments may be inserted. Another type of vector is a viral vector, wherein additional DNA segments may be inserted into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell and thereby are replicated along with the host genome. In addition, certain vectors are capable of directing the expression of genes they comprise. Such vectors are referred to herein as “expression vectors”.

In a fourth aspect, the present invention relates to a cell comprising the one or more polynucleotides according to the second aspect of the invention or the one more expression vectors according to the third aspect of the invention. A wide variety of cell expression systems can be used to express said polynucleotides including the use of prokaryotic and eukaryotic cells, such as bacterial cells (e.g. E. coli), yeast cells, insect cells or mammalian cells (e.g. mouse cells, rat cells, human cells etc.). For this purpose, a cell is transformed or transfected with said polynucleotide(s) or expression vector(s) such that the polynucleotide(s) of the invention are expressed in the cell and, in one embodiment, secreted into the medium in which the cells are cultured, from where the expression product can be recovered.

### CH2 Domains With Internal Disulfide Bonds

In another aspect, the present invention provides a CH2 domain, wherein said CH2 domain comprises an internal disulfide bond which is not present in the corresponding wild-type CH2 domain. In some embodiments, the present invention provides an IgG1 CH2 domain, wherein said CH2 domain comprises an internal disulfide bond which is not present in the wild-type IgG1 CH2 domain. In some embodiments, said internal disulfide bond in the IgG1 CH2 domain is selected from the group consisting of disulfide bonds formed between cysteine residues at the following positions according to EU numbering: P238C and D265C; P238C and A327C; P238C and L328C; S267C and A327C; and V240C and I332C. In some embodiments, said internal disulfide bond in the IgG1 CH2 domain is selected from the group consisting of disulfide bonds formed between cysteine residues at the following positions: P238C and L328C; S267C and A327C; and V240C and I332C. In some embodiments, said internal disulfide bond in the IgG1 CH2 domain is a disulfide bond formed between cysteine residues P238C and D265C. In some embodiments, said internal disulfide bond is a disulfide bond formed between cysteine residues P238C and A327C. In some embodiments, said internal disulfide bond in the IgG1 CH2 domain is a disulfide bond formed between cysteine residues P238C and L328C. In some embodiments, said internal disulfide bond in the IgG1 CH2 domain is a disulfide bond formed between cysteine residues S267C and A327C. In some embodiments, said internal disulfide bond is a disulfide bond formed between cysteine residues V240C and I332C.

In some embodiments, the present invention provides a CH2 domain, wherein said CH2 domains comprises at least two internal disulfide bonds which are not present in the corresponding wild-type CH2 domain. In some embodiments, the present invention provides an IgG1 CH2 domain, wherein said CH2 domains comprises at least two internal disulfide bonds which are not present in the wild-type IgG1 CH2 domain. In some embodiments, said internal disulfide bonds in the IgG1 CH2 domain are selected from the group consisting of disulfide bonds formed between cysteine residues at the following positions according to EU numbering: P238C and D265C; P238C and A327C; P238C and L328C; S267C and A327C; and V240C and I332C. In some embodiments, said internal disulfide bonds in the IgG1 CH2 domain are formed between cysteine residues at the following positions: P238C and D265C as well as S267C and A327C; P238C and D265C as well as V240C and I332C; P238C and A327C as well as V240C and I332C; P238C and L328C as well as S267C and A327C; P238C and L328C as well as V240C and I332C; S267C and A327C as well as V240C and I332C.

It is also possible to introduce additional disulfide bonds to stabilize the molecule, e.g. intradomain disulfide bonds in the CH3 domain as described in the art (Wozniak-Knopp PLOS One. 2012; 7 (1): e30083).

In some embodiments, the CH2 domain which comprises an internal disulfide bond which is not present in the corresponding wild-type CH2 domain is part of a larger molecule. In some embodiments, the molecule is an antibody. In some embodiments, the molecule is a multispecific antibody. In some embodiments, the molecule is a monospecific antibody.

### Examples

Example 1 shows deals with the anti-TNF antibody adalimumab, while Example 2 employs a GLP1 receptor agonist (RA) Fc fusion protein.

### Methods

The following section summarizes the methods which were used in the Examples below.

### Protein Constructs and Protein Expression

All proteins were transiently expressed in CHO or HEK293 cells. The sequences of the different antibody variants are shown in the sequence listing. The DNAs for the different protein chains were synthesized (Thermo Fisher Scientific) and cloned into an expression vector under a CMV promotor sequence and a leader sequence required for proper protein secretion into the culture supernatant. Expression and purification were done essentially as described by Becker et al. (2019) Protein Expr Purif. 2019 January; 153:1-6. In brief, cells were transfected using PEI in a ratio of DNA: PEI of 1:3. Expression was done for 6 days and cells were separated from the culture supernatant by centrifugation (20 min, 3000 g, 4° C.). Culture supernatants were 0.22 μm filtered and loaded onto A MabSelect Sure column (GE Healthcare) equilibrated in phosphate buffered saline (PBS, Gibco/Thermo Fisher Scientific). After elution, proteins were desalted and further purified using a Superdex 200 (GE Healthcare) size exclusion chromatography column equilibrated with PBS (Gibco).

The sequences for the GLP1-RA Fc fusion proteins were either directly flanked by a leader sequence or a leader sequence containing a His-tag followed by a Tev-cleavage site. For purification via the His-tag, proteins were captured on a His-tag purification resin (complete, Roche) column, equilibrated in 300 mM NaCl, 50 mM Tris, pH 8.0. Column was washed with 300 mM NaCl, 50 mM Tris, pH 8.0, 5 mM imidazole and the protein was eluted with 300 mM NaCl, 50 mM Tris, pH 8.0, 500 mM imidazole. Tev-cleavage was done at room temperature in 100 mM NaCl, 50 mM, 50 mM Tris, pH 8.0 overnight and the cleavage mixture was directly loaded on a protein A column and further purified. When a His-tag was not present, the proteins were purified using protein A as described above.

### SDS-PAGE Analysis

Protein samples (5 μg protein) were mixed with either 4×LDS sample buffer (Life Technologies/Thermo Fisher Scientific) or 4×LDS sample buffer with 50 mM dithiothreitol for SDS-PAGE analysis under non-reducing or reducing conditions, respectively. Samples were incubated for 5 min at 99° C. before loading on a 4-12% SDS-PAGE with MES as running buffer (Life Technologies/Thermo Fisher Scientific). BenchMark protein ladder was used as marker (Invitrogen/Thermo Fisher Scientific).

### Analytical Size Exclusion Chromatograph and Multi-Angel Static Light Scattering Measurements (SEC-MALS)

Analytical size exclusion chromatography (SEC) runs were done using a Superdex 200 10/HR column (GE Healthcare) equilibrated in freshly degassed and 0.2 μm filtered PBS (Gibco). Sample volume was 50 μl for all samples and protein concentration was adjusted to 3 mg/ml for all samples. An ÄKTA purifier (GE Healthcare) was used as chromatography system. The system was connected to a multi-angle static light scattering (MALS) detector (miniDawn, Wyatt) and a refractive index (RI)-detector (Shodex RI-101, Showa-Denko) as concentration detector. Data analysis was done using the ASTA 5.3.4.20 (Wyatt) software. A refractive index increment (dn/dc) value of 0.185 was used for all samples.

### MS-Analysis

Protein integrity was checked by LC-MS. Protein samples were deglycosylated with 12.5 μg of protein diluted to 0.5 mg/ml in ddH₂O containing PNGaseF (1:50 v/v) (glycerol free, New England Biolabs) at 37° C. for 15 h. If samples were analysed under reduced conditions 0.1 M dithiothreitol was added. The LC-MS analysis was done on an Agilent 6540 Ultra High Definition (UHD) Q-TOF equipped with a dual ESI interface and an Agilent 1290/1260 Infinity LC System. Reversed phase (RP) chromatography was done using a PLRP-S 1000A 5 μm, 50×2.1 mm (Agilent) with a guard column PLRP-S 300A 5 μm, 3×5 mm (Agilent) at 200 μL/min and 80° C. column temperature.

1 μg protein was injected onto the column and eluted with a linear gradient from 0 to 17 min with increasing acetonitrile concentration. Eluents used were buffer A containing LC water and 0.1 formic acid and buffer B containing 90% acetonitrile, 10% LC water and 0.1 formic acid. Obtained mass data were analysed using the software MassHunter Bioconfirm B.06 (Agilent). Molecular masses were calculated based on the amino acid sequences of the proteins using GPMAW software version 10.32 (Lighthouse Data, Denmark).

### Surface Plasmon Resonance Analysis

All analysis of antibody binding to FcRs were performed on a Biacore T200 instrument (GE Healthcare). FcγR binding assays were done using anti-His capture. Anti-tetra His (Qiagen) was buffer exchanged into PBS pH 7.2 (Gibco), diluted to 25 μg/mL in 10 mM sodium acetate pH 4.0 and directly immobilized to a series S CM5 chip to a surface density of ˜10,000 RU using the amine coupling kit provided by GE Healthcare. His-tagged recombinant human and mouse FcγRs (FcγRI/CD64 #1257-FC; FcγRIIa/CD32a (R167) #1330-CD; FcγRIIb/c/CD32b/c #1875-CD; FcγRIIIa/CD16a #4325-FC; FcγR4/CD16-2 #4325-FC; R&D Systems) were diluted to 2 μg/mL in HBS-EP+ (10 mM HEPES pH 7.4, 150 mM NaCl, 3 mM EDTA, 0.05% surfactant P20) and injected for 30 sec at 10 μL/min flowrate. Antibodies were serially diluted 3-fold from 3000 to 37 nM and injected over the captured receptors for 2 min in duplicate. For binding to hFcγRI, serial dilutions were made from 300 nM to 3.7 nM. The surface was regenerated with 10 mM glycine pH 1.5 for 30 sec. Sensorgrams were processed using the BiaEvaluation software (GE Healthcare) and fit to a 1:1 binding model to obtain kinetic constants. FcRn binding measurements were done using biotinylated FcRn. Biotinylated human FcRn protein was purchased from Immunitrack (#ITF01). A capture assay using a biotin capture kit (GE Healthcare) for reversible capture of the biotinylated FcRn was used. The CAP chip surfaces were prepared by injecting the biotin capture reagent solution for 300 sec at a flow rate of 2 μL/min. The FcRn protein was captured at a concentration of 0.5 μg/mL and a flow rate of 10 μL/min for 90 sec to reach a capture level of typically 100 RU. The analyzed proteins were used in a 1:1 dilution series in HBS-EP+buffer from 1600 nM to 12.5 nM. The proteins were injected for 60 sec and dissociation by buffer inject was measured for 120 sec. HBS-EP+ buffer as used as assay buffer at a flow rate of 30 μl/min. Chip surfaces were regenerated with the regeneration solution of the biotin capture kit as described by the manufacturer. Binding curves were analyzed in the BIAcore T200 evaluation software v2.0 using a flow cell without captured FcRn as reference and subtraction of a buffer inject (double referencing). The equilibrium dissociation constants (KD) were determined by global steady state data fitting of the equilibrium binding responses (Req) for each protein concentration.

Binding of human TNFα to antibodies was performed using surface plasmon resonance on a Biacore 3000 instrument (GE Healthcare). The antibodies were immobilized on the chip and TNFα was used as analyte. For capture of the antibodies the human antibody capture kit (GE Healthcare) was used. The capture antibody was immobilized via primary amine groups (typically 10,000 RU) on a research grade CM5 chip (GE Life Sciences) using standard procedures as described for the human antibody capture kit. The analyzed antibody was captured at a flow rate of 10 μL/min with an adjusted RU value that would result in maximal analyte binding signal of typically 30 RU. Binding traces were measured against recombinant human TNFα (Sigma-Aldrich, #H8916) at a concentration of 100 nM with association and dissociation times of 240 sec and 300 sec, respectively. HBS-EP was used as assay buffer at a flow rate of 30 μl/min. Chip surfaces were regenerated with the regeneration solution of the human antibody capture kit. Binding traces were analyzed in the BIAevaluation program package v4.1 using a flow cell without captured antibody as reference and subtraction of a buffer inject (double referencing).

### ADCC and ADCD In Vitro Assays

The target cells were generated by transfecting a CHO-K1 cell line with a mutant form of human TNFα. This mutant form is known to be only poorly cleaved by membrane proteases and remains at the cell surface. This TNFα-form was cloned into a mammalian expression vector under the CMV promotor further containing a neomycine resistance gene for selection. Cells were grown in Ham's F12 medium (Biowest, Nuaillé, France) containing 10% heat-inactivated fetal bovine serum (FBS, Gibco) and 1 mg/mL G418 antibioticum (Invivogen, Toulouse, France) for selection. Both assays use the same target cell and are based on the chromium (⁵¹Cr) release assay. For ADCD assays, target cells were washed with PBS and incubated with trypsin for 4 minutes. Cells were labelled with ⁵¹Cr, washed and plated in 96 well plates at approx. 3000 cells per well and incubated with RPM1-1640 (Biowest) medium containing 5% FBS. Guinea pig complement (Standard complement, Tebubio) at a fixed concentration and antibodies were added at different concentrations and further incubated for 4 hours at 37° C. For radioactivity measurements, aliqots were withdrawn, transferred into a LumaPlate (PerkinElmer) and gamma counting was done using a MicroBeta Jet device (PerkinElmer). ADCC assays were performed in a similar manner as described for the ADCD assay but instead of adding complement, effector cells were added at a ratio of 10:1 (effector:target cell). The used effector cell were monoclonal human cytotoxic T lymphocytes expressing the human FcγRIIIa (V158) receptor as described in the literature (Clémenceau et al., Blood. 2006; 107 (12):4669-4677). In all assays, negative controls were included were neither complement or effector cells, respectively, were added (spontaneous release). Maximum ⁵¹Cr release was achieved by adding 0.75% Triton X-100 to the cell culture medium. Results are expressed as % specific release and is calculated following the equation: % specific release=(experimental release−spontaneous release)/(maximum release−spontaneous release). All measurements were done in triplicates.

### Pharmacodynamics In Mice

The mice studies were performed at Covance (Covance Laboratories Inc, Greenfield, IN). All procedures were in compliance with the U.S. Department of Agriculture's (USDA) Animal Welfare Act (9 CFR Parts 1, 2, and 3) and the Guide for the Care and Use of Laboratory animals (Institute for Laboratory Animal Research.

Female db/db (BKS.Cgo−+Leprdb/+Leprdb/OlaHsd) or lean mice (BKS.Cg−[lean]/OlaHsd) were from Envigo. Animals were 12 weeks old at study start and grouped with n=8 animals. Mice were feed with Purina 5008 ad libitum, had free access to water and were maintained on a 12 h light/dark cycle. As a stratification criterion HbA1c values (hemolysate method) were determined at day 7 of the predose phase. HbA1c is a is a biomarker for the average blood glucose level during the preceding ˜4 weeks. Low, medium and high HbA1c values were spread as equally as possible across groups resulting in similar Hb1Ac group-means. All animals were treated once every 4 days s.c. with an application volume of 5 mL/kg with vehicle (PBS, Gibco Ref 14190) or compound at 10 nmol/kg. Blood samples were withdrawn from the tail tip without anaesthesia and blood glucose was measured with a glucometer on day 9 of the predose-phase and days 1, 5, 9 and 13 (0 and 4 hr postdose) as well as days 2, 3, 4, 6, 7, 8, 10, 11, 12, and 14 of the dosing phase. Body weights were measured daily.

### GLP1-Receptor Activity Assays

Agonism of compounds for human glucagon-like peptide-1 (GLP1) receptor was determined by functional assays measuring CAMP response of recombinant PSC-HEK-293 cell line stably expressing human GLP1 receptor. Cells were grown in a T175 culture flask placed at 37° C. to near confluence in medium (DMEM/10% FBS) and collected in 2 ml vials in cell culture medium containing 10% DMSO in concentration of 1-5×10⁷ cells/ml. Each vial contained 1.8 ml cells. The vials were slowly frozen to −80° C. in isopropanol, and then transferred in liquid nitrogen for storage. Prior to their use, frozen cells were thawed quickly at 37° C. and washed (5 min at 900 rpm) with 20 ml cell buffer (1×HBSS; 20 mM HEPES, plus 0.1% HSA if indicated in example conditions). Cells were resuspended in assay buffer (cell buffer plus 2 mM IBMX) and adjusted to a cell density of 1×106 cells/ml. For measurement, 5 μl cells (final 5000 cells/well) and 5 μL of test compound were added to a 384-well plate, followed by incubation for 30 minutes at room temperature. The CAMP content of cells was determined using homogenous time resolved fluorescence technique. (HTFR, CisBio). After addition of HTRF reagents diluted in lysis buffer (kit components), the plates were incubated for 1 h, followed by measurement of the fluorescence ratio at 665/620 nm. In vitro potency of agonists was quantified by determining the concentrations that caused 50% activation of maximal response (EC50).

### Thermal Stability Measurements

Analysis of thermal stability was done using a Prometheus NT Flex device (NanoTemper Technologies) based on the nanoDSF technology. The device is equipped with Aggregation Optics allowing collection of scattering information simultaneously with fluorescence measurements. Measurements were done in the range from 20° C. to 95° C. with a thermal ramp of 1° C./min following the instructions of the manufacturer. All protein samples were dissolved in PBS and protein concentration was adjusted to 0.5 mg/mL. Measurements were done in duplicates. Data analysis was done using the software PR ThermControl V.2.1 (NanoTemper Technologies).

### Example 1: Generation of Fc Variants of Adalimumab According to the Present Invention

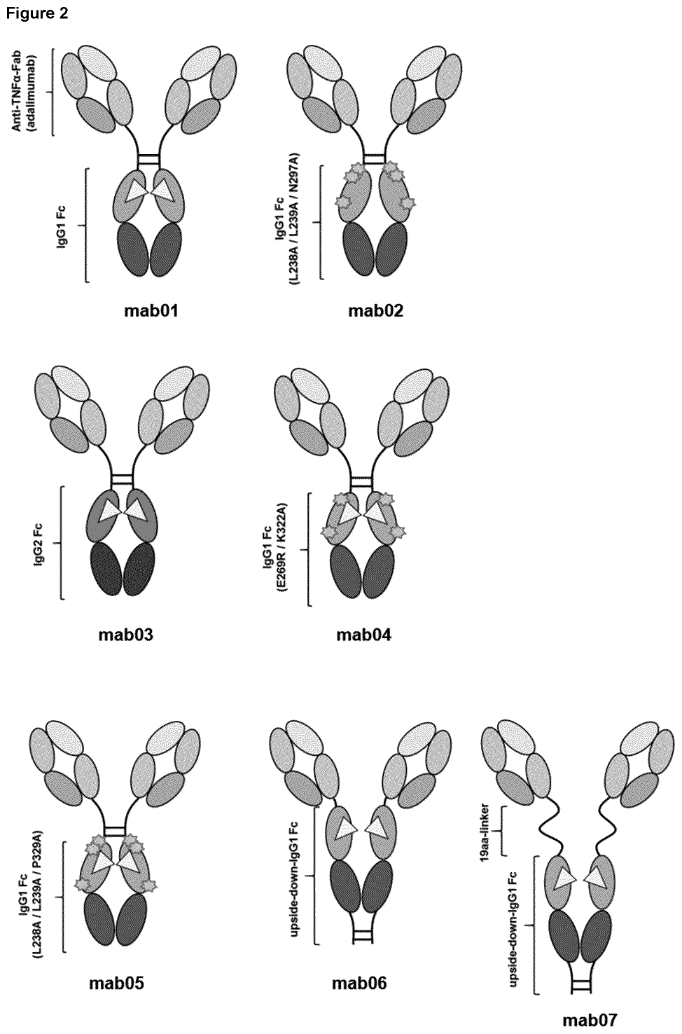

The concept of Fc molecules according to the present invention was tested for the anti-TNF antibody adalimumab. We created two different versions of an Fc adalimumab according to the present invention (FIG. 2, mab06 (heavy chain SEQ ID NO: 10) and mab07 (heavy chain SEQ ID NO: 11)). These two constructs differed only in the length of the linker replacing the hinge region. For comparison purposes, Fc-backbone variants known to have silenced effector functions were made as well (FIG. 2, mab02, mab04, mab05). The Fab was the same in all constructs. All antibodies were transiently expressed in CHO or Hek293 cells and purified using a two-step purification procedure. Interestingly, the highest yields were obtained with the Fc variants according to the present invention (Tab. 1).

TABLE 1
Obtained yields (after two-step purification) for the
different antibody variants.

| Fc-backbone | Yield [mg/L] |
|---|---|
| IgG1 | |
| IgG2 | 100.4 |
| udIgG1 | 200.7 |
| udIgG1 (19 GS linker) | 150.6 |
| IgG1 LALA N297A | 120.7 |
| IgG1 LALA P239A | 90.4 |
| IgG1 E269R, K322A | 140.7 |

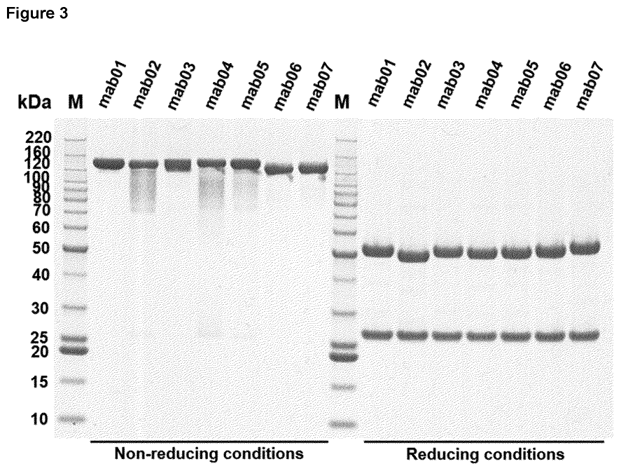

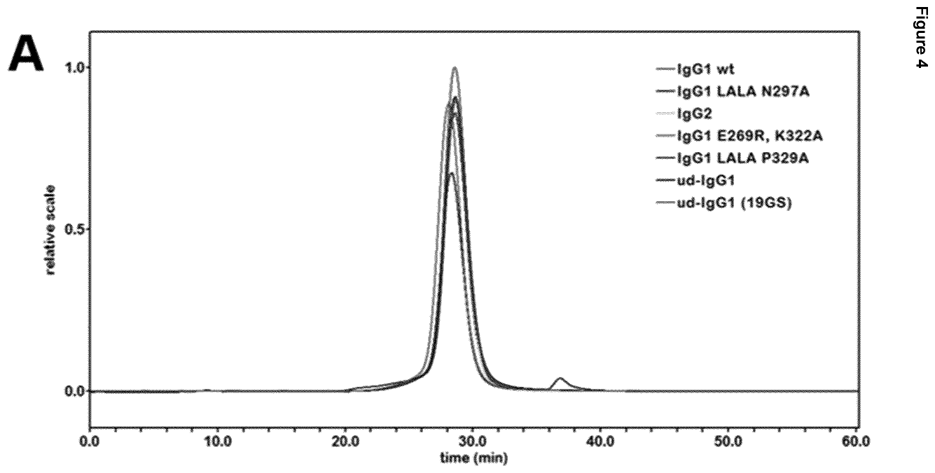

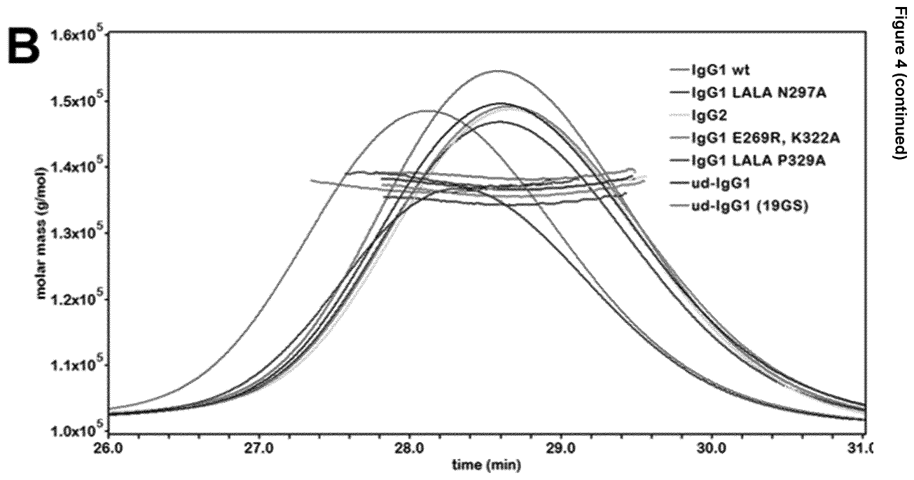

All antibodies were analyzed for homogeneity by SDS-PAGE (FIG. 3) and analytical size exclusion chromatography coupled with MALS. Light scattering measurement is a very sensitive method for the detection of large particle, e.g. aggregates. Furthermore, it allows the calculation of molecular masses without the need for reference molecules. In all antibody samples the presence of aggregates was below the detection limit (FIG. 4). The obtained calculated molecular masses for all antibodies was in good accordance with the calculated values. In addition, identity of all antibody variants was verified by MS (Tab. 2).

TABLE 2
Calculated masses and masses obtained by SEC-MALS
and MS for each antibody.
Molecular masses were calculated with GPMAW
(average mass values). All cysteines were
assumed to form disulfide-bridges.

| | Calculated Mw | Mw by SEC- | Mw by MS |
|---|---|---|---|
| Antibody | (Da) non-reduced | MALS (Da) | (intact) (Da) |
| mab01 | 145,189 | 138,700 | 145,197 |
| mab02 | 144,935 | 134,800 | 144,940 |
| mab03 | 144,743 | 137,300 | 144,751 |
| mab04 | 145,129 | 136,200 | 145,138 |
| mab05 | 144,969 | 137,900 | 144,976 |
| mab06 | 145,409 | 137,200 | 145,418 |
| mab07 | 147,785 | 136,800 | 147,793 |

The Fc IgG1 variant according to the present invention with 19GS-linker (mab07) elutes slightly earlier from the SEC column. In this molecule, the Fab domains are connected via long linker (GGGGGGGGGGGGSGGGGSA, SEQ ID NO: 12) to the Fc region. This arrangement obviously allows a more open molecule configuration resulting in a slightly enhanced hydrodynamic radius.

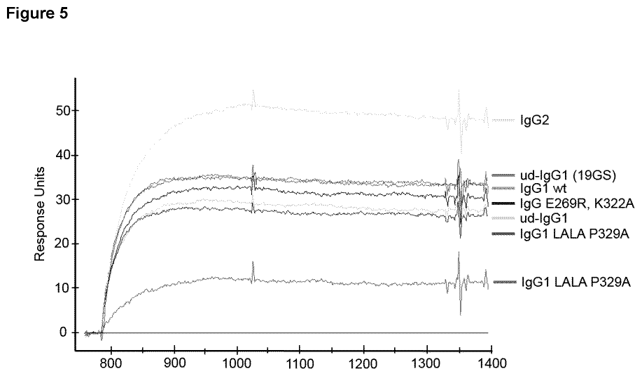

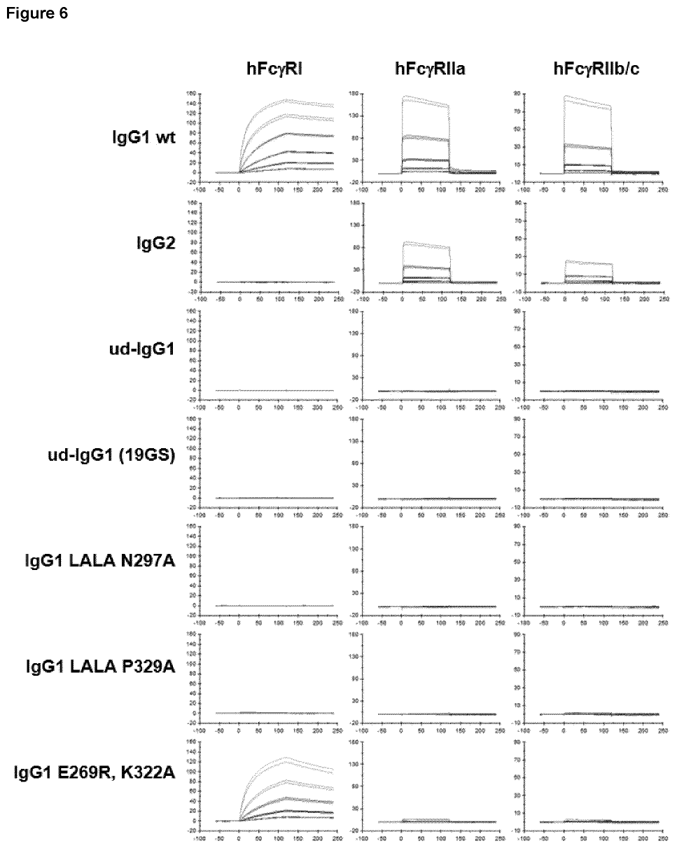

Binding to the antigen, human TNFα, was checked by surface plasmon resonance-measurements (FIG. 5).

### Binding Assays to FcRs

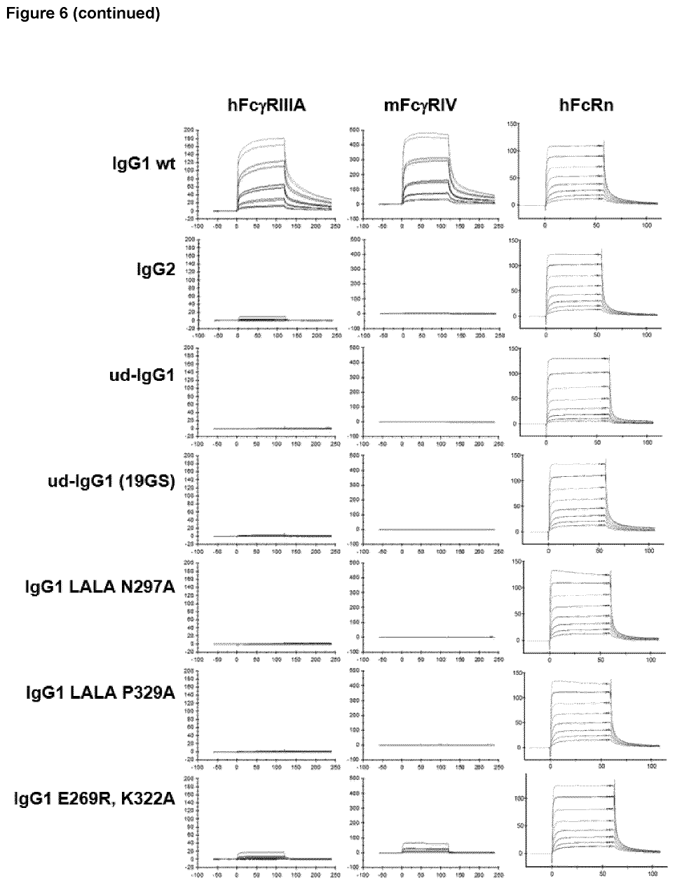

The Fc variants of adalimumab according to the present invention lack the FcγR binding site located in the IgG hinge region (FIG. 1). Thus, they should not bind to FcγRs. To check whether binding of the Fc variants according to the present invention to the FcγRs was indeed abolished we measured binding to immobilized FcγRs using surface plasmon resonance in comparison to other antibody variants carrying silencing mutations. We analyzed binding to human FcγRI, FcγRIIa, b and c, and FcγRIIIa. Moreover, we included the murine activating FcγRIV since many antibodies and Fc fusion proteins are analyzed in mouse models. Finally, the human FcRn receptor, which is important for recycling of antibodies and Fc fusion proteins, was also included in the test panel. The results are summarized in Tab. 3 and the corresponding sensorgrams are shown in FIG. 6.

TABLE 3
Affinity measurements of the different anti-TNFα-antibodies to different human
FcγRs. Kds were determined, where possible, using a 1:1 kinetic model (n/d:
no binding detected; *too low: signal was too low to obtain binding constants).

| | hFcγRI | hFcγRIIa(R167) | hFcγRIIb/c | hFcγRIIIa(V158) | mFcγRIV | hFcRn |
|---|---|---|---|---|---|---|
| Antibody | Kd [nM] | Kd [nM] | Kd [nM] | Kd [nM] | Kd [nM] | Kd [nM] |
| mab01 | 0.8 | 3280 | >10000 | 656 | 827 | 361 |
| mab02 | n/d | n/d | n/d | n/d | n/d | 263 |
| mab03 | *too low | >5000 | >10000 | >10000 | >10000 | 342 |
| mab04 | 6.7 | *too low | n/d | >10000 | >5000 | 357 |
| mab05 | >10000 | n/d | n/d | n/d | n/d | 265 |
| mab06 | n/d | n/d | n/d | n/d | n/d | 528 |
| mab07 | *too low | n/d | n/d | n/d | n/d | 348 |

The unmodified IgG1 backbone (mab01) binds to all FcγR. The highest binding affinity was obtained with FcγRI, which is known as a high affinity binding receptor. An unmodified IgG2 (mab03), tested for comparison purposes, shows some binding to FcRγIIa, FcγRIIb/c and to a lower extent to FcRγIIIa. The antibodies with the Fc IgG1 according to the present invention (mab06, mab07) showed no detectable binding to all tested FcγRs. This is also true for the IgG1 LALA N297A (mab02) and IgG1 LALA P329A (mab05) backbone variants. For the IgG1 E269R, K322A variant (mab04) a high affinity binding to FcγRI could still be measured. Binding of this variant to the FcγRIIIa and murine FcγRIV was significantly reduced but still detectable. All analyzed Fc variants show similar binding affinity for the hFcRn.

### Functional Cellular Assays (ADCC and ADCD)

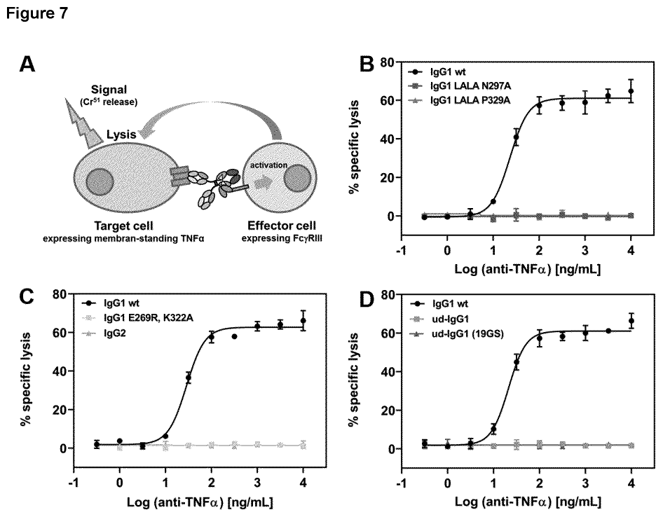

ADCC and ADCD are the most important mechanisms for the effector functions of antibodies. Thus, we wanted to ensure that the Fc variants of adalimumab according to the present invention do not elicit ADCC or ADCD. In an ADCC assay the target cells expressing the membrane-standing antigen is incubated with the antibody of interest and effector cells. If an effector cell is tethered to the target cell it gets activated and kills the target cell (FIG. 7A). All analyzed antibody variants show no detectable specific target cell lysis, while the IgG1 wt variant provokes significant specific cell lysis (FIG. 7B-D).

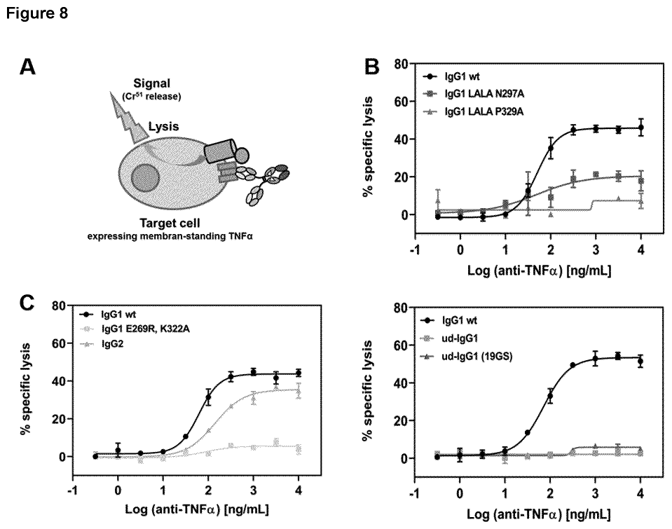

The ADCD mechanism leads to cell death by activation of the complement system. The complement cascade is triggered by binding of the C1q protein to antigen-antibody complexes on the cell surface of the target cell. The final step of complement cascade activation is the formation of the so-called membrane attack complex which leads to cell lysis (FIG. 8A). The IgG1 LALA N297A (mab02) and the IgG2 backbone (mab03) are still able to induce significant specific cell lysis (FIG. 8B, C). The IgG1 E269R, K322A variant (mab04) triggered a lower but still detectable specific cell lysis. For the Fc IgG1 variants according to the present invention (mab06, mab07) and the IgG1 LALA P329A variants (mab05) no or no significant specific cell lysis was observed (FIG. 8B, D).

### Examples 2: GLP1-Receptor-Agonist Fc-Domain Fusion Proteins

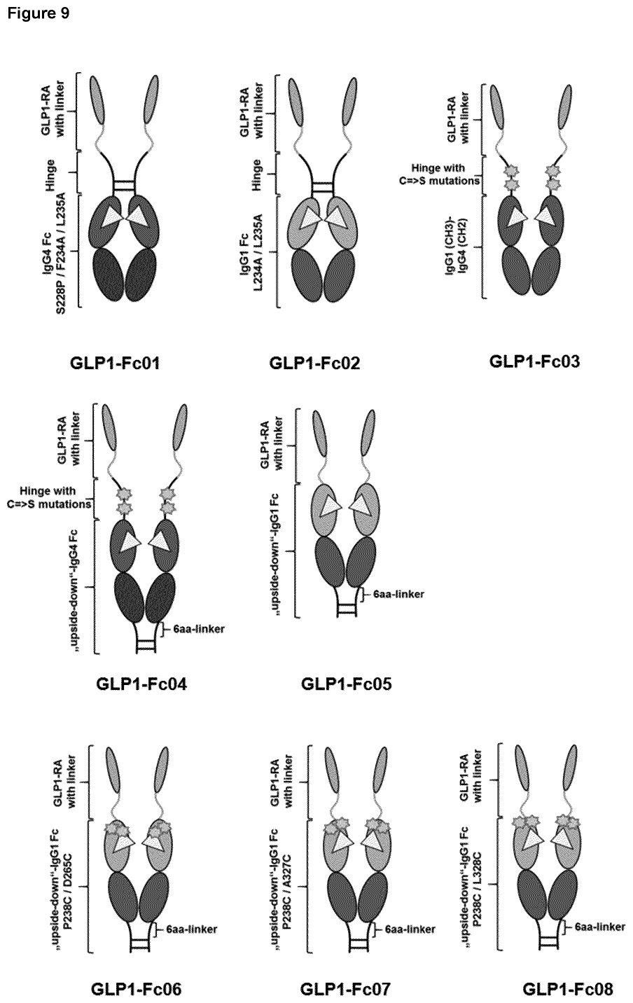

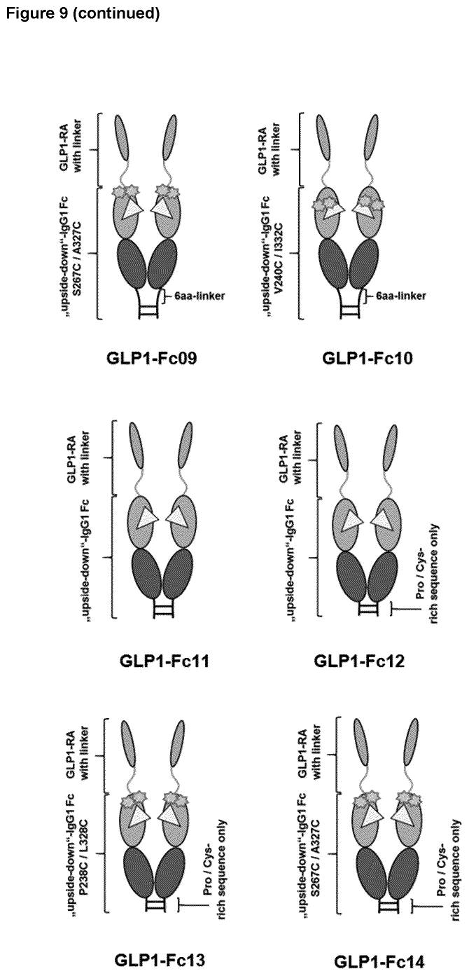

To evaluate the Fc format according to the present invention for a peptide Fc fusion protein and to test in vivo functionality, we used GLP1-receptor agonist (RA) Fc fusion proteins. Thus, we fused a human GLP1-RA via a peptide linker to an IgG4 Fc region (FIG. 9, GLP1-Fc01 and 04) or to an IgG1 Fc region FIG. 9, GLP1-Fc02, 05-014). FIG. 9 shows an overview of the different GLP1-RA-Fc fusion protein variants.

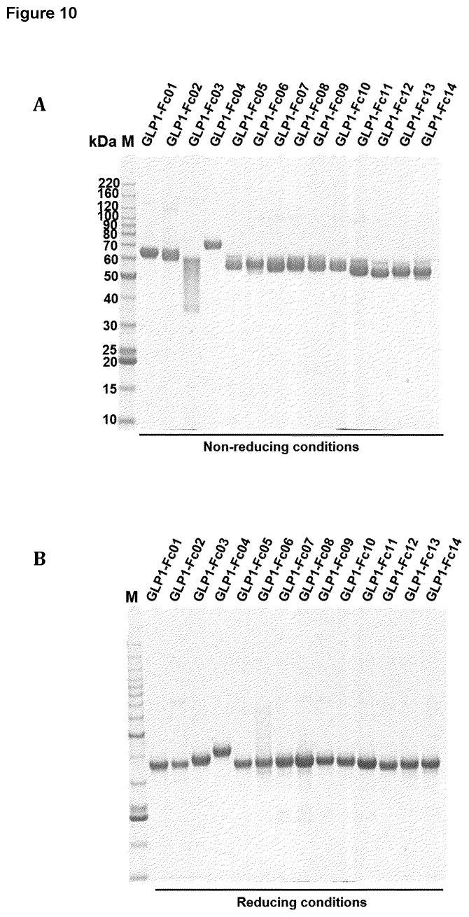

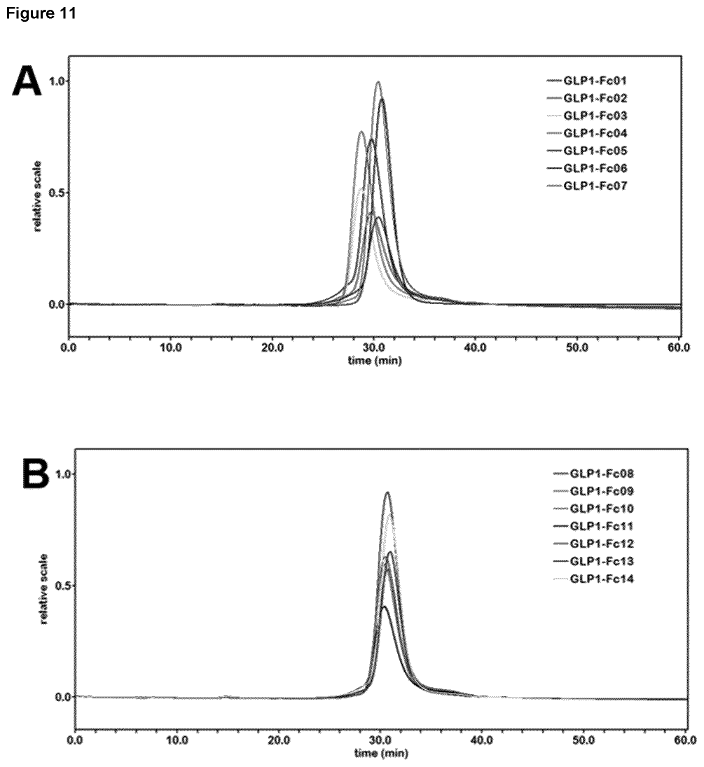

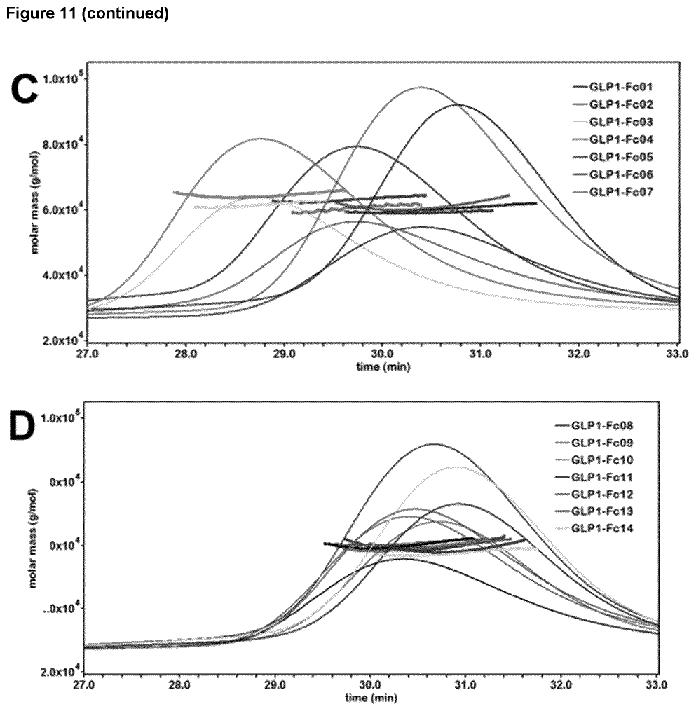

In addition to simple Fc variants according to the present invention for IgG4 (GLP1-Fc04) and IgG1 (GLP1-Fc05), we created 9 Fc variants according to the present invention for IgG1 (GLP1-Fc06-14) which comprise an additional internal disulfide bond in the CH2 domain. This is intended to additionally stabilize the CH2 domain since it is known that the IgG CH2 domain unfolds earlier than the IgG CH3 domain. While 6 of the variants with an additional internal disulfide bond had a sequence derived from the wild-type hinge region sequence at the C-terminus (GLP-Fc06-11), the other 3 variants had a shortened proline-cysteine rich sequence at the C-terminus (GLP-Fc12-14). All GLP1-Fc-RA fusion proteins were analyzed for homogeneity by SDS-PAGE (FIG. 10) and analytical size exclusion chromatography coupled with MALS (FIG. 11). In all analyzed samples, presences of aggregates was below detection limit and the obtained molecular weight were in good accordance with the calculated molar mass of a homo-dimeric molecule (Tab. 4).

TABLE 4
Calculated masses and masses obtained by SEC-MALS and MS for
GLP1-Fc fusion proteins. Molecular masses were calculated with
GPMAW (average mass values). All cysteines
were assumed to form disulfide-bridges.

| | Calculated | Mw by | Mw by MS | Calculated | Mw |
|---|---|---|---|---|---|
| | Mw | SEC- | non- | Mw | by MS |
| | non-reduced | MALS | reduced | reduced | reduced |
| Protein | (Da) | (Da) | (Da) | (Da) | (Da) |
| GLP1-Fc01 | 59,670 | 62,690 | 59,675 | 29,841 | 29,840 |
| GLP1-Fc02 | 60,382 | 60,560 | n.d. | 30,193 | 30,194 |
| GLP1-Fc03 | 59,608 | 61,580 | n.d. | 29,808 | 29,808 |
| GLP1-Fc04 | 63,770 | 64,480 | n.d. | 31,891 | 31,891 |
| GLP1-Fc05 | 59,545 | 59,180 | n.d. | 29,772 | n.d. |
| GLP1-Fc06 | 59,533 | 60.620 | n.d. | 29,766 | n.d. |
| GLP1-Fc07 | 59,621 | 60,013 | n.d. | 29,810 | n.d. |
| GLP1-Fc08 | 59,537 | 59,750 | n.d. | 29,768 | n.d. |
| GLP1-Fc09 | 59,641 | 60,014 | n.d. | 29,820 | n.d. |
| GLP1-Fc10 | 59,533 | 60,790 | n.d. | 29,766 | n.d. |
| GLP1-Fc11 | 58,914 | 60,150 | n.d. | 29,457 | n.d. |
| GLP1-Fc12 | 57,757 | 59,400 | n.d. | 28,878 | n.d. |
| GLP1-Fc13 | 57,749 | 58,900 | n.d. | 28,874 | 28,871 |
| GLP1-Fc14 | 57,853 | 57,750 | n.d. | 28,926 | 28,925 |

N.d.: not determined

For comparison purposes, we included also a variant without any covalent bond between the heavy chains (GLP1-Fc03). In contrast to the other tested variants, this GLP1-Fc03 variant showed a clear dissociation in the SDS-PAGE under non-reducing conditions.

The activity for all variants was checked in a GLP1-R activation assay. To ensure that the introduced disulfide-bonds do not interfere with binding to the FcRn, binding to FcRn was checked using surface plasmon resonance technology at pH 6.0. GLP1 activity data and binding data to FcRn are shown in Tab. 5.

TABLE 5
Activity data and binding data to the FcRn. EC50
values were determined with a
GLP1R expressing reporter cell line. Binding to the
human FcRn was measured using surface
plasmon resonance technique and thermal stability
measurements were done with nanoDSF.

| | EC50 | EC50 | hFcRn | mFcRn |
|---|---|---|---|---|
| | hGLP1R | mGLP1R | (pH6.0) | (pH6.0) |
| Variant | [pMol] | [pMol] | Kd [nM] | Kd [nM] |
| GLP1-Fc01 | 2.08 | 1.47 | 514 | 306 |
| GLP1-Fc02 | 4.12 | n.d. | 637 | n.d. |
| GLP1-Fc03 | 1.69 | 0.84 | 315 | 146 |
| GLP1-Fc04 | 2.21 | 0.99 | 500 | 233 |
| GLP1-Fc05 | 2.59 | n.d. | 451 | n.d. |
| GLP1-Fc06 | 102.8 | n.d. | 363 | n.d. |
| GLP1-Fc07 | 3.19 | n.d. | 519 | n.d. |
| GLP1-Fc08 | 4.16 | n.d. | 558 | n.d. |
| GLP1-Fc09 | 4.87 | n.d. | 868 | n.d. |
| GLP1-Fc10 | 2.75 | n.d. | 862 | n.d. |
| GLP1-Fc11 | 2.95 | n.d. | 814 | n.d. |
| GLP1-Fc12 | 2.40 | n.d. | 497 | n.d. |
| GLP1-Fc13 | 0.48 | n.d. | n.d. | n.d. |
| GLP1-Fc14 | 0.18 | n.d. | n.d. | n.d. |
| GLP1 (ctrl) | 0.58 | 0.71 | n.d. | n.d. |

n.d.: not determined.

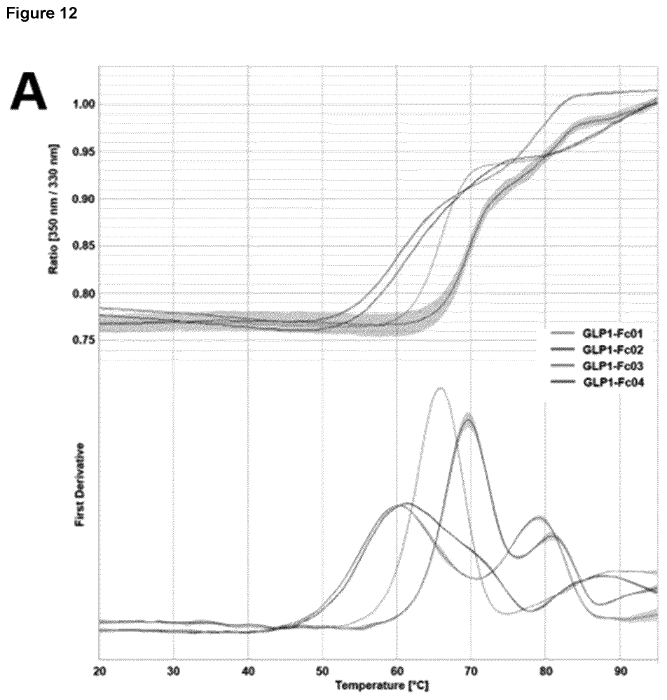

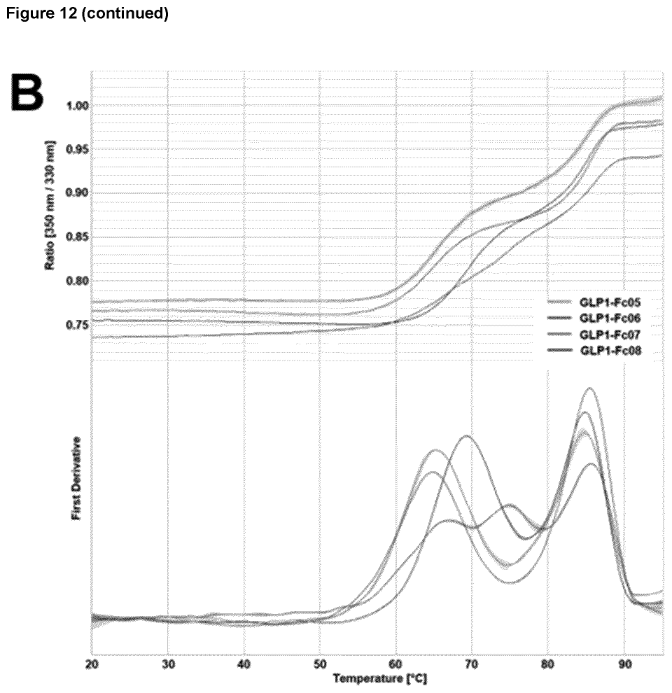

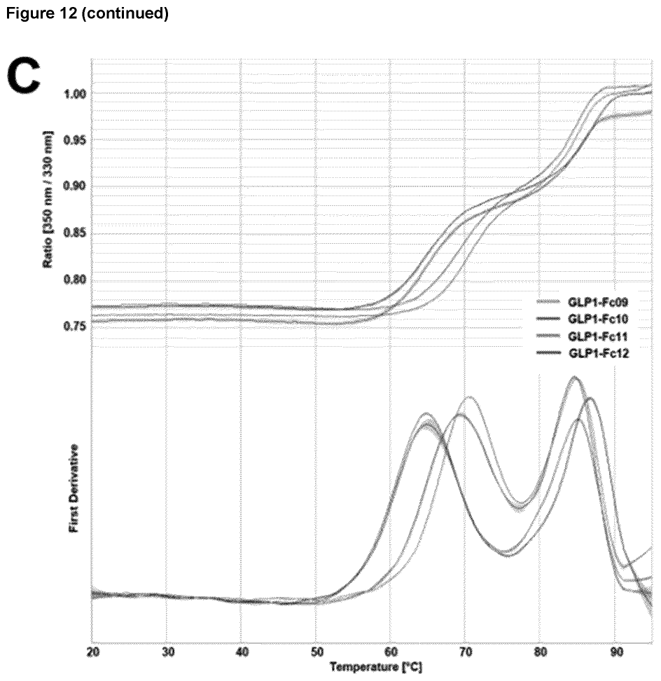

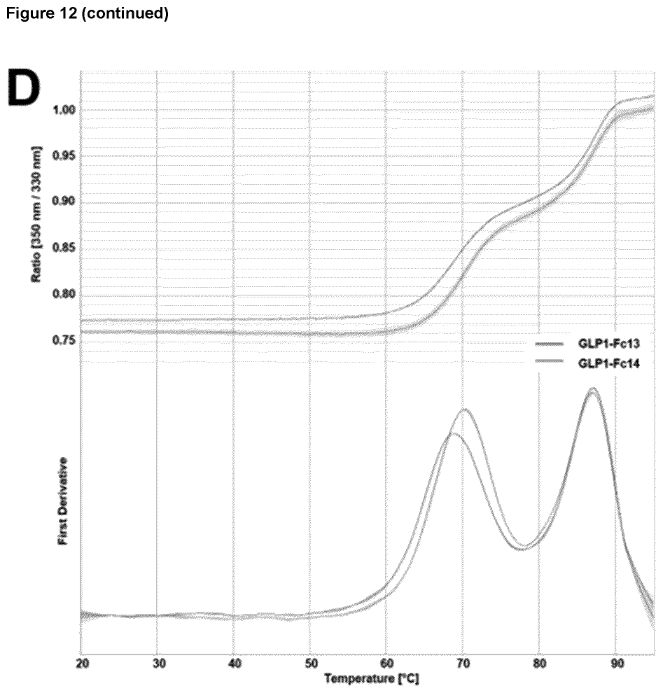

Thermal stability of GLP1-RA-Fc fusion proteins was analyzed with the DSF method (FIG. 12, Tab. 6).

TABLE 6
Stability of GLP1-RA-Fc variants as determined with
nanoDSF. Data analyzing was
done using standard procedures with the software PR
Stability Analysis V.1.0.3. Ton is the onset
temperature at which proteins begin to unfold. The
inflection points (IP) are the maxima from the
first derivative of the melting curves. All data were
measured in duplicates, shown are mean data.

| Variant | Tₒₙ [° C.] | IP #1 [° C.] | IP #2 [° C.] | IP#3 [° C.] |
|---|---|---|---|---|
| GLP1-Fc01 | 57.39 | 65.90 | 88.92 | — |
| GLP1-Fc02 | 61.90 | 69.62 | 80.72 | — |
| GLP1-Fc03 | 47.20 | 60.26 | 78.95 | — |
| GLP1-Fc04 | 48.41 | 61.39 | 70.84 | 87.87 |
| GLP1-Fc05 | 55.86 | 65.36 | 84.74 | — |
| GLP1-Fc06 | 57.05 | 67.12 | 74.79 | 85.53 |
| GLP1-Fc07 | 53.47 | 64.86 | 85.40 | — |
| GLP1-Fc08 | 59.65 | 69.35 | 84.76 | — |
| GLP1-Fc09 | 61.02 | 70.63 | 84.88 | — |
| GLP1-Fc10 | 58.88 | 69.43 | 84.60 | — |
| GLP1-Fc11 | 54.42 | 64.94 | 85.04 | — |
| GLP1-Fc12 | 54.81 | 64.99 | 86.66 | — |
| GLP1-Fc13 | 59.34 | 69.00 | 87.01 | — |
| GLP1-Fc14 | 60.75 | 70.28 | 86.97 | — |

GLP1-Fc01 (the unmodified IgG4 backbone) shows one major unfolding event. GLP1-Fc02 (the unmodified IgG1 backbone) resulted in a more stabilized protein and a second unfolding event is clearly detectable. GLP1-Fc03 (the variant without any covalent bond between the heavy chains) is more thermolabile than GLP1-Fc01 and GLP1-Fc02. GLP1-Fc04 (Fc IgG4 variant according to the present invention) is slightly more stable than GLP1-Fc03. In GLP1-Fc02 and GLP1-Fc03 the CH3 domain is derived from IgG1 whereas in GLP-Fc01 and GLP1-Fc04 the CH3 domain is from IgG4. It is evident, that a clear second unfolding event is only measured in variants with an IgG1 derived CH3 domain (FIG. 12A). Based on these data, the IgG1 Fc backbone was chosen for further engineering. An internal disulfide-bond was introduced into the CH2 domain to further improve stability in the Fc variants according to the present invention GLP1-Fc06-GLP1-Fc10. While GLP1-Fc06 showed a slightly higher thermostability and GLP-Fc07 showed no stabilization at all, the introduced disulfide bond in GLP1-Fc08, GLP1-Fc09, and GLP1-Fc10 resulted in proteins with comparable stability as the IgG1 LALA Fc backbone (GLP1-Fc-02; FIG. 12, Tab. 6).

To further stabilize the Fc variants according to the present invention, the linker sequence between the Fc region and the C-terminal sequence corresponding to the hinge region was omitted (GLP1-Fc11). In another approach, the C-terminal sequence corresponding to the hinge region was shortened to a proline-cysteine rich sequence (GLP1-Fc12). For these proteins, an increase in thermostability for the second unfolding event (CH3 domain) was achieved (FIG. 12C, Tab. 6). The stabilizing effects of an internal disulfide bond within the CH2 domain and a shortened proline-cysteine rich sequence are combined in GLP1-Fc13 and GLP1-Fc14 (FIG. 12D, Tab. 6).

### Functionality In Vivo

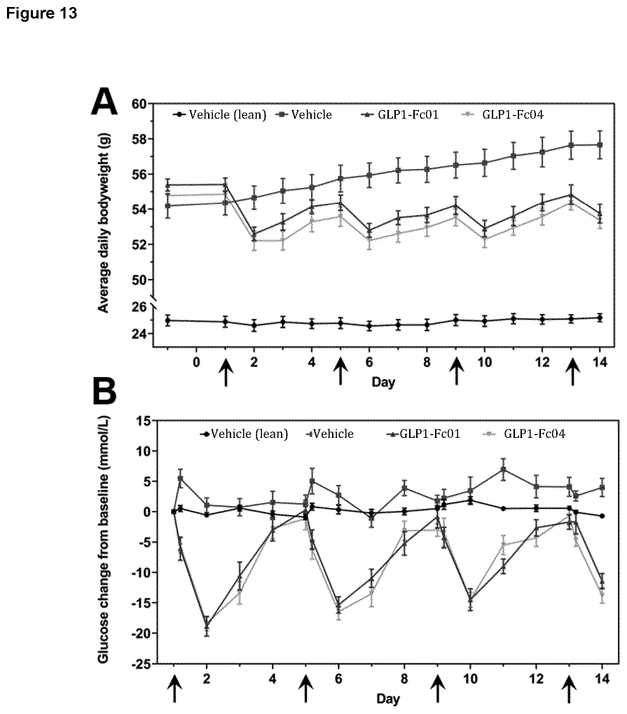

In GLP1-Fc04 the original hinge was not deleted but the cysteines were mutated to serines. This was done to have an Fc variant of the GLP1-RA Fc fusion protein according to the present invention which is as similar as possible to the parent molecule and this variant was chosen for a head-to-head pharmacodynamic analysis. Both proteins were administered s.c. every fourth day in db/db-mice for 14 days and blood glucose as well as body weight was monitored (FIG. 13). Mean body weights at day −1 (n=8 for each group) were 24.9 g (lean vehicle control), 54.2 g (db/db vehicle control), 55.4 g (GLP1-Fc01 group), and 54.8 g (GLP1-Fc04 group). After the treatment period mean body weights were 25.2 g (lean vehicle control, +1.2%), 57.7 g (db/db vehicle control, +6.5%), 53.8 g (GLP1-Fc01 group, −2.9%), and 50.4 g (GLP1-Fc04 group, −2.6%). For the lean vehicle control body weight remained nearly constant over the treatment period whereas the db/db vehicle control gained body weight. Both treated groups lost body weight at a comparable range. Both compounds, GLP1-Fc01 and GLP1-Fc04, show undistinguishable effects on the change in blood glucose levels. These findings suggest that the GLP1-RA maintains its full activity when fused to the Fc variant according to the present invention. The coincident profile of blood glucose change over the time suggest, that the recycling function of the Fc variant according to the present invention is not compromised in vivo.

### Disclosed items

Disclosed is also the Following:
1. A molecule comprising two polypeptides,
   wherein said polypeptides form an antibody Fc region,
   wherein said two polypeptides are linked by at least two covalent bonds which are located C-terminally to the portion of each polypeptide which forms the Fc region,
   wherein said two polypeptides are not linked by a disulfide bond located N-terminally to the portion of each polypeptide which forms the Fc region.
2. The molecule of claim 1, wherein the two covalent bonds are two disulfide bonds.
3. The molecule of any of the preceding claims, wherein the at least two covalent bonds located C-terminally to the portion of each polypeptide which forms the Fc region are comprised in a sequence corresponding to the amino acid sequence of an antibody hinge region.
4. The molecule of any of the preceding claims, wherein the affinity to C1q and/or FcγR1, FcγR2 and/or FcγR3 is reduced at least 10-fold, compared to the same molecule comprising an antibody hinge region located N-terminally to the portion of each polypeptide which forms the Fc region.
5. The molecule of any of the preceding claims, wherein the affinity to protein A and/or FcgRn is not reduced, compared to the same molecule comprising an antibody hinge region located N-terminally to the portion of each polypeptide which forms the Fc region.
6. The molecule of any of the preceding claims, wherein the Fc region is the Fc region of IgG, IgM, IgA, IgD or IgE.
7. The molecule of any of the preceding claims, wherein the at least two covalent bonds are located C-terminally to a CH2, CH3 and/or CH4 domain on each polypeptide.
8. The molecule of any of the proceeding claims, wherein the Fc region is the Fc region of an IgG antibody.
9. The molecule of any of the preceding claims, wherein the at least two covalent bonds are located C-terminally to a CH2 and/or CH3 domain on each polypeptide.
10. The molecule of any of the preceding claims, wherein the at least two covalent bonds are located C-terminally to a CH2 and CH3 domain on each polypeptide.
11. The molecule of any of the preceding claims, wherein each polypeptide comprises a CH2 domain which is part of the Fc region, wherein each of these CH2 domains comprises an internal disulfide bond which is not present in the corresponding wild-type CH2 domain.
12. The molecule of claim 11, wherein the additional internal disulfide bond is selected from the group consisting of disulfide bonds formed between cysteine residues at the following positions according to EU numbering: P238C and D265C; P238C and A327C; P238C and L328C; S267C and A327C; and V240C and I332C.
13. The molecule of any of the preceding claims, wherein each of the polypeptides comprises a linker, wherein the linker links the C-terminus of portion of the polypeptide forming the Fc region to the at least two covalent bonds located C-terminally to said portion of the polypeptide forming the Fc region.
14. The molecule of any of the preceding claims, wherein the molecule further comprises at least one active moiety.
15. A polynucleotide encoding a molecule according to any of the preceding claims.

1. A molecule comprising two polypeptides,
wherein said polypeptides form an antibody Fc region,
wherein said two polypeptides are linked by at least two covalent bonds which are located C-terminally to the portion of each polypeptide which forms the Fc region,
wherein said two polypeptides are not linked by a disulfide bond located N-terminally to the portion of each polypeptide which forms the Fc region,
wherein each polypeptide comprises a CH2 domain which is part of the Fc region, wherein each of these CH2 domains comprises an additional internal disulfide bond which is not present in the corresponding wild-type CH2 domain,
wherein the additional internal disulfide bond is selected from the group consisting of disulfide bonds formed between cysteine residues at the following positions according to EU numbering: P238C and D265C; P238C and L328C; S267C and A327C; and V240C and I332C.

2. The molecule of claim 1, wherein the additional internal disulfide bond is selected from the group consisting of disulfide bonds formed between cysteine residues at the following positions according to EU numbering: P238C and L328C; S267C and A327C; and V240C and I332C.

3. The molecule of claim 1, wherein the additional internal disulfide bond is selected from the group consisting of disulfide bonds formed between cysteine residues at the following positions according to EU numbering: P238C and L328C; and S267C and A327C.

4. The molecule of claim 1, wherein the two covalent bonds are two disulfide bonds.

5. The molecule of claim 1, wherein the at least two covalent bonds located C-terminally to the portion of each polypeptide which forms the Fc region are comprised in a sequence corresponding to the amino acid sequence of an antibody hinge region.

6. The molecule of claim 1, wherein the affinity to C1q and/or FcγR1, FcγR2 and/or FcγR3 is reduced at least 10-fold, compared to the same molecule comprising an antibody hinge region located N-terminally to the portion of each polypeptide which forms the Fc region.

7. The molecule of claim 1, wherein the affinity to protein A and/or FcgRn is not reduced, compared to the same molecule comprising an antibody hinge region located N-terminally to the portion of each polypeptide which forms the Fc region.

8. The molecule of claim 1, wherein the Fc region is the Fc region of IgG, IgM, IgA, IgD or IgE.

9. The molecule of claim 1, wherein the at least two covalent bonds are located C-terminally to a CH3 or CH4 domain on each polypeptide.

10. The molecule of claim 1, wherein the Fc region is the Fc region of an IgG antibody.

11. The molecule of claim 1, wherein the at least two covalent bonds are located C-terminally to a CH3 domain on each polypeptide.

12. The molecule of claim 1, wherein the molecule further comprises at least one active moiety.

13. The molecule of claim 12, wherein the active moiety is located N-terminal of the CH2 domain of at least one polypeptide and the molecule does not comprise an active moiety which is located C-terminal of the at least two covalent bonds which are located C-terminally to the portion of each polypeptide which forms the Fc region.

14. The molecule of claim 12, wherein the active moiety is an antigen-binding moiety.

15. A polynucleotide encoding a molecule according to claim 1.
